(19) United States
(12) Patent Application Publication
(10) Pub. No.: US 2026/0166139 A1
(43) Pub. Date: Jun. 18, 2026

(54) Anti-CTLA4 Monoclonal Antibody or its Antigen Binding Fragments, Pharmaceutical Compositions and Uses

(71) Applicant: AKESO BIOPHARMA, INC., Zhongshan (CN)

(72) Inventors: Baiyong Li, Zhongshan (CN); Yu Xia, Zhongshan (CN); Zhongmin Wang, Zhongshan (CN); Peng Zhang, Zhongshan (CN); Xinghua Pang, Zhongshan (CN)

(73) Assignee: AKESO BIOPHARMA, INC., Zhongshan (CN)

(21) Appl. No.: 19/334,022

(22) Filed: Sep. 19, 2025

Related U.S. Application Data
(63) Continuation of application No. 17/695,592, filed on Mar. 15, 2022, now Pat. No. 12,447,208.
Application No. 17/695,592 is a continuation of application No. 16/562,236, filed on Sep. 5, 2019, now Pat. No. 11,291,720.
Application No. 16/562,236 is a continuation of application No. 15/500,744, now Pat. No. 10,449,251.

(30) Foreign Application Priority Data
Aug. 1, 2014 (CN) 201410377352.9

(51) Int. Cl. A61K 39/395; C07K 16/28; C07K 19/00; C12N 5/10; C12N 15/63; G01N 33/577

(52) U.S. Cl. CPC A61K 39/39541; A61K 39/395; C07K 16/28; C07K 16/2896; C07K 19/00; C12N 5/10; C12N 15/63; G01N 33/577

(57) ABSTRACT

The present invention belongs to the fields of tumor therapy and molecular immunology, and provides an anti-CTLA4 monoclonal antibody or antigen binding fragment thereof, a pharmaceutical composition thereof and use thereof. The monoclonal antibody of the present invention can block the binding of CLTA4 to B7, relieve the immunosuppression on the body by CTLA4, and activate T lymphocytes.

### CROSS-REFERENCE TO RELATED APPLICATIONS

This application is a continuation of U.S. patent application Ser. No. 17/695,592, filed Mar. 15, 2022, now U.S. Pat. No. 12,447,208, issued Oct. 21, 2025, which is a continuation of U.S. patent application Ser. No. 16/562,236, filed Sep. 5, 2019, now U.S. Pat. No. 11,291,720, issued Mar. 16, 2022, which is a continuation of U.S. patent application Ser. No. 15/500,744, filed Jan. 31, 2017, now U.S. Pat. No. 10,449,251, issued Oct. 2, 2019, which is the National Phase Application of International Application No. PCT/CN2015/085721, filed Jul. 31, 2015, which claims priority from Chinese Patent Application No. CN 201410377352.9, filed Aug. 1, 2014.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created Sep. 23, 2025, is named 00238-US-CNT-3_CL.xml and is 51,322 bytes in size.

### TECHNICAL FIELD

The present invention belongs to the fields of tumor therapy and molecular immunology. The present invention relates to an anti-CTLA4 monoclonal antibody or antigen binding fragment thereof, a pharmaceutical composition thereof, encoding sequences thereof and a method of and use in diagnosis, prevention, therapy and/or adjuvant therapy using the same.

### TECHNICAL BACKGROUND

Cytotoxic T lymphocyte associated antigen 4 (abbreviated as CTLA4) has very close relationship with the CD28 molecule in gene structure, chromosome location, sequence homology and gene expression. Both of them are receptors for the co-stimulative molecule B7, mainly expressed on the surface of activated T cells. However, as co-stimulating signal of lymphocyte activation, CTLA4 has opposite function to CD28. After binding to B7, CTLA4 can inhibit the activation of mouse and human T cells, playing a negative regulating role in the activation of T cells.

CTLA4 mAbs or CTLA4 ligands can prevent CTLA4 from binding to its native ligands, thereby blocking the transduction of the T cell negative regulating signal by CTLA4 and enhancing the responsivity of T cells to various antigens. In this aspect, results from in vivo and in vitro studies are substantially in concert. At present, there are some CTLA4 mAbs (10D1, 11.2.2) being tested in clinical trials for treating prostate cancer, bladder cancer, colorectal cancer, cancer of gastrointestinal tract, liver cancer, malignant melanoma, etc (CTLA-4 blockade in tumor models: an overview of preclinical and translational research. Grosso J F., Jure-Kunkel M N., Cancer Immun. 2013; 13:5. Epub 2013 Jan. 22; U.S. Pat. No. 6,984,720 B1; and U.S. Pat. No. 6,682,736 B1). Among them, 10D1 and 11.2.2 are regarded as among those anti-CTLA4 monoclonal antibodies having best effects.

Interleukin 2 (IL-2) is produced by T cells. It is a growth factor regulating a subgroup of T cells. It is also an important factor modulating immune response. It can promote and activate the expansion of B cells, and involves in antibody reaction, hematopoiesis and tumor surveillance. Recombinant human IL-2 has been approved by US FDA for the treatment of malignant tumors (including melanoma, kidney tumor, etc). It is also under clinical studies of treating chronic viral infection (Pharmacologic administration of interleukin-2. Chavez, A. R., et al., Ann N Y Acad Sci, 2009. 1182: 14-27).

As important factors affecting the function of T cells, CTLA4 and CTLA4 mAbs can produce specific therapeutic effect on diseases by interfering with the immune microenvironment in the body. They have high efficacy and remedy the deficiency of traditional medication, opening a novel pathway of gene therapy. CTLA4 and CTLA4 mAbs are being tested in experiments and various stages of clinical trials. For example, in autoimmune diseases, they effectively inhibited airway hyperresponsiveness in an animal model of asthma, prevented the development of rheumatic diseases, mediated immune tolerance to an allograft in the body, and the like. On the other hand, although biological gene therapy has not shown any adverse effect in short term clinical trials, attention should be paid to the potential effect after long term application. For example, excessive blockade of CTLA4 bound B7 signaling by CTLA4 mAbs may result in the development of autoimmune diseases. As antibodies can specifically bind to their ligands and induce the lysis of target cells or block the progress of pathology, development and utilization of drugs based on antibodies, especially humanized antibodies have important significance in the clinical treatment of malignant tumors and other immune diseases in humans.

At present, there is yet a need to develop novel antibodies blocking the binding of CTLA4 to B7, and their humanized antibodies.

### SUMMARY OF THE INVENTION

After intensive studies and creative works by the inventors, recombinant CTLA4 was expressed using a mammal cell expression system, and used as the antigen to immunize mice. Hybridoma cells were obtained by fusing the mouse splenic cells with myeloma cells. After screening a great number of samples by the inventors, a hybridoma cell line capable of secreting and producing a specific monoclonal antibody which specifically binds CTLA4 and can block the binding of CTLA4 to B7 very effectively, was obtained. Furthermore, humanized antibodies were generated. Thus, the following inventions are provided.

One aspect of the present invention relates to a monoclonal antibody or antigen binding fragment thereof, wherein the monoclonal antibody comprises the complementary determining regions (CDR's) selected from the following:
HCDR1 comprising the amino acid sequence of SEQ ID NO: 27,
HCDR2 comprising the amino acid sequence of SEQ ID NO: 28, and
HCDR3 comprising the amino acid sequence of SEQ ID NO: 29;
and/or
LCDR1 comprising the amino acid sequence of SEQ ID NO: 30,
LCDR2 comprising the amino acid sequence of SEQ ID NO: 31, and
LCDR3 comprising an amino acid sequence selected from SEQ ID NO: 32, SEQ ID NO: 33, and SEQ ID NO: 34.

The monoclonal antibody or antigen binding fragment thereof according to any one of the embodiments of the present invention, wherein
the amino acid sequence of the heavy chain variable region (VH) of the monoclonal antibody is selected from SEQ ID NO: 6, SEQ ID NO: 10, SEQ ID NO: 14, and SEQ ID NO: 18; and/or
the amino acid sequence of the light chain variable region (VL) of the monoclonal antibody is selected from SEQ ID NO: 8, SEQ ID NO: 12, SEQ ID NO: 16, SEQ ID NO: 20, SEQ ID NO: 22, and SEQ ID NO: 24.

The monoclonal antibody or antigen binding fragment thereof according to any one of the embodiments of the present invention, wherein the monoclonal antibody comprises
(1) VH as set forth in SEQ ID NO: 6 and VL as set forth in SEQ ID NO: 8;
(2) VH as set forth in SEQ ID NO: 10 and VL as set forth in SEQ ID NO: 12;
(3) VH as set forth in SEQ ID NO: 14 and VL as set forth in SEQ ID NO: 16;
(4) VH as set forth in SEQ ID NO: 18 and VL as set forth in SEQ ID NO: 20;
(5) VH as set forth in SEQ ID NO: 14 and VL as set forth in SEQ ID NO: 22; or
(6) VH as set forth in SEQ ID NO: 14 and VL as set forth in SEQ ID NO: 24.

In the present invention, the above groups (1) to (6) show the amino acid sequences of the heavy chain variable region and light chain variable region of 8D2/8D2(Re), 8D2H1L1, 8D2H2L2, 8D2H3L3, 8D2H2L15, and 8D2H2L17, respectively.

Specifically, the methionine (Met) at position 18 in SEQ ID NO: 6, SEQ ID NO: 10, and SEQ ID NO: 14 is independently substituted with an amino acid selected from the following: Leucine (Leu), Valine (Val), Isoleucine (Ile), or Alanine (Ala).

Therapeutic drugs based on antibodies, especially monoclonal antibodies (MAB) have achieved excellent efficacy in the treatment of several diseases. The traditional method to obtain such therapeutic antibody is immunizing an animal with an antigen, obtaining antibodies against the antigen from the immunized animal, or improving an antibody having low affinity to the antigen by affinity maturation. However, such method is time consuming and labor consuming, and often fails to target a specific epitope on the antigen.

Antigen binding is dependent on the variable regions of the light chain and heavy chain; the variable region of each chain comprises three hypervariable regions, also called complementarity determining region (CDR) (the heavy chain (H) comprises HCDR1, HCDR2 and HCDR3, and the light chain (L) comprises LCDR1, LCDR2 and LCDR3; for definition, see Kabat et al., Sequences of Proteins of Immunological Interest, Fifth Edition (1991), Vol. 1-3, NIH Publication 91-3242, Bethesda Md).

The amino acid sequences of the CDRs in the monoclonal antibody sequences of the embodiments (1) to (6) above were analyzed by technical means well known to those skilled in the art, e.g., by VBASE2 database analysis. The results are provided below.

(1) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

(2) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

(3) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

(4) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

(5) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

(6) The amino acid sequences of the 3 CDRs of the heavy chain variable region are shown below:

HCDR1:
(SEQ ID NO: 27)
GFTFSDNW,
HCDR2:
(SEQ ID NO: 28)
IRNKPYNYET,
HCDR3:
(SEQ ID NO: 29)
TAQFAY.

The amino acid sequences of the 3 CDRs of the light chain variable region are shown below:

LCDR1:
(SEQ ID NO: 30)
ENIYGG,
LCDR2:
(SEQ ID NO: 31)
GAT,
LCDR3:
(SEQ ID NO: 32)
QNVLRSPFT.

The monoclonal antibody or antigen binding fragment thereof according to any one of the embodiments of the present invention, wherein the monoclonal antibody or antigen binding fragment thereof is selected from an Fab, an Fab′, an F(ab′)2, an Fd, an Fv, a dAb, a complementarity determining region fragment, a single chain antibody (e.g., an scFv), a humanized antibody, a chimeric antibody, or a diabody.

The monoclonal antibody or antigen binding fragment thereof according to any one of the embodiments of the present invention, wherein the monoclonal antibody binds the CTLA4 protein with a KD less than about 10⁻⁵ M, e.g., less than about 10⁻⁶ M, 10⁻⁷ M, 10⁻⁸ M, 10⁻⁹ M, or 10⁻¹⁰ M or less.

The monoclonal antibody or antigen binding fragment thereof according to any one of the embodiments of the present invention, wherein the monoclonal antibody comprises non-CDR regions, and said non-CDR regions are from an antibody of a species other than murine, e.g., a human.

The monoclonal antibody or antigen binding fragment thereof of the present invention is an anti-CTLA4 monoclonal antibody or antigen binding fragment thereof that can specifically bind CTLA4.

The monoclonal antibody or antigen binding fragment thereof according to any one of the embodiments of the present invention for use in the prevention and/or therapy and/or adjuvant therapy and/or diagnosis of a tumor; specifically, the tumor is selected from melanoma, kidney tumor/renal tumor, prostate cancer, bladder cancer, colorectal cancer, cancer of gastrointestinal tract, and liver cancer/hepatic cancer.

The monoclonal antibody according to any one of the embodiments of the present invention for use in:
blocking the binding of CTLA4 to B7,
regulating (e.g., down-regulating) the activity of CTLA4 or the level of CTLA4,
relieving the immunosuppression on the body by CTLA4, or
an agent activating T lymphocytes or increasing the expression of IL-2 in T lymphocytes.

Another aspect of the present invention relates to an isolated nucleic acid molecule, which comprises a nucleic acid sequence capable of encoding the heavy chain variable region of an antibody, wherein
the heavy chain variable region of the antibody comprises CDRs with the amino acid sequences of SEQ ID NOs: 27-29;
specifically, the heavy chain variable region of the antibody has the amino acid sequence as set forth in SEQ ID NO: 6, SEQ ID NO: 10, SEQ ID NO: 14, or SEQ ID NO: 18;
more specifically, the nucleic acid molecule has the nucleotide sequence as set forth in SEQ ID NO: 5, SEQ ID NO: 9, SEQ ID NO: 13, or SEQ ID NO: 17.

The present invention further provides isolated nucleic acid molecules, which encode the monoclonal antibody or antigen binding fragment thereof of the present invention. Such nucleic acid molecules can be isolated from the hybridoma cells, or obtained through recombinant technologies of gene engineering or methods of chemical synthesis.

A further aspect of the present invention relates to an isolated nucleic acid molecule, which comprises a nucleic acid sequence capable of encoding the light chain variable region of an antibodies, wherein the light chain variable region of the antibody comprises
1) CDRs with the amino acid sequences of SEQ ID NOs: 30-32;
2) CDRs with the amino acid sequences of SEQ ID NO: 30, SEQ ID NO: 31 and SEQ ID NO: 33; or
3) CDRs with the amino acid sequences of SEQ ID NO: 30, SEQ ID NO: 31 and SEQ ID NO: 34;
specifically, the light chain variable region of the antibody has the amino acid sequence as set forth in SEQ ID NO: 8, SEQ ID NO: 12, SEQ ID NO: 16, SEQ ID NO: 20, SEQ ID NO: 22, or SEQ ID NO: 24;
more specifically, the nucleic acid molecule has the nucleotide sequence as set forth in SEQ ID NO: SEQ ID NO: 7, SEQ ID NO: 11, SEQ ID NO: 15, SEQ ID NO: 19, SEQ ID NO: 21, or SEQ ID NO: 23.

A further aspect of the present invention relates to a vector, which comprises the isolated nucleic acid molecule according to any one the embodiments of the present invention. The vector of the present invention can be a cloning vector or an expression vector. In a preferred embodiment, the vector of the present invention is, for example, a plasmid, a cosmid, a bacteriophage, a coemid, or the like.

A further aspect of the present invention relates to a host cell, which comprises the isolated nucleic acid molecule of any one of the embodiments of the present invention, or the vector according to the present invention. Such host cells include, but are not limited to, prokaryotic cells such as E. coli cells, and eukaryotic cells such as yeast cells, insect cells, plant cells, and animal cells (such as mammalian cells, including mouse cells, human cells, or the like). The cells of the present invention can also be a cell line, such as 293T cells.

A further aspect of the present invention relates to a method of preparing the monoclonal antibody or antigen binding fragment thereof according to any one of the embodiments of the present invention, the method comprising the steps of culturing the host cell of the present invention under suitable conditions and recovering the monoclonal antibody or antigen binding fragment thereof from the cell culture.

A further aspect of the present invention relates to a conjugate, which comprises a monoclonal antibody or antigen binding fragment thereof and a conjugated moiety, wherein the monoclonal antibody is a monoclonal antibody or antigen binding fragment thereof according to any one of the embodiments of the present invention, and the conjugated moiety is a detectable label. Specifically, the conjugated moiety is a radioisotope, a fluorescent substance, a luminescent substance, a chromogenic substance, or an enzyme (e.g., horse radish peroxidase).

A further aspect of the present invention relates to a kit, which comprises the monoclonal antibody or antigen binding fragment thereof according to any one of the embodiments of the present invention, or the conjugate according to the present invention; specifically, the kit further comprises a second antibody, which specifically recognizes said monoclonal antibody or antigen binding fragment thereof; optionally, said second antibody further comprises a detectable label, e.g., a radioisotope, a fluorescent substance, a luminescent substance, a chromogenic substance, or an enzyme (e.g., horse radish peroxidase).

A further aspect of the present invention relates to use of the monoclonal antibody or antigen binding fragment thereof according to any one of the embodiments of the present invention in the preparation of a kit for use in detecting the presence or level of CTLA4 in a sample.

A further aspect of the present invention relates to a pharmaceutical composition, which comprises the monoclonal antibody or antigen binding fragment thereof according to any one of the embodiments of the present invention or the conjugate according to the present invention; optionally, it further comprises a pharmaceutically acceptable carrier and/or excipient.

A further aspect of the present invention relates to use of the monoclonal antibody or antigen binding fragment thereof according to any one of the embodiments of the present invention or the conjugate according to the present invention in the preparation of a medicament for use in the prevention and/or therapy and/or adjuvant therapy and/or diagnosis of a tumor; specifically, the tumor is selected from melanoma, kidney tumor/renal tumor, prostate cancer, bladder cancer, colorectal cancer, cancer of gastrointestinal tract, and liver cancer/hepatic cancer.

A further aspect of the present invention relates to use of the monoclonal antibody or antigen binding fragment thereof according to any one of the embodiments of the present invention or the conjugate according to the present invention in the preparation of a following agent:
an agent that detects the presence or level of CTLA4 in a sample,
an agent that blocks the binding of CTLA4 to B7,
an agent that regulates (e.g., down-regulates) the activity of CTLA4 or the level of CTLA4,
an agent that relieves the immunosuppression on the body by CTLA4,
an agent that activates T lymphocytes, or
an agent that increases the expression of IL-2 in T lymphocytes.

A further aspect of the present invention relates to an in vivo or in vitro method comprising the step of administrating to cells an effective amount of the monoclonal antibody or antigen binding fragment thereof according to any one of the embodiments of the present invention or the conjugate according to the present invention, wherein the method is selected from the following:
a method of detecting the presence or level of CTLA4 in a sample,
a method of blocking the binding of CTLA4 to B7,
a method of regulating (e.g., down-regulating) the activity of CTLA4 or the level of CTLA4,
a method of relieving the immunosuppression on the body by CTLA4,
a method of activating T lymphocytes, or
a method of increasing the expression of IL-2 in T lymphocytes.

Said methods can be used for diagnostic or therapeutic purposes, or non-diagnostic or non-therapeutic purposes (e.g., where the sample is a cell sample, rather than a sample from a patient).

A further aspect of the present invention relates to a method of the prevention and/or therapy and/or adjuvant therapy and/or diagnosis of a tumor, comprising administrating to a subject an effective amount of the monoclonal antibody or antigen binding fragment thereof according to any one of the embodiments of the present invention or the conjugate according to the present invention; specifically, the tumor is selected from melanoma, kidney tumor/renal tumor, prostate cancer, bladder cancer, colorectal cancer, cancer of gastrointestinal tract, and liver cancer/hepatic cancer.

In the present invention, unless stated otherwise, scientific and technical terms used herein have the same meaning as commonly understood by those skilled in the art. Moreover, procedures of cell culture, molecular genetics, nucleic acid chemistry, immunology used herein are the widely utilized methodologies in the relevant art. Meanwhile, for purpose of better understanding the present invention, the definitions and explanations of relevant terms are provided below.

As used herein, when reference is made to the amino acid sequence of the CTLA4 protein (Cytotoxic T-Lymphocyte Antigen 4), it includes the full length of the CTLA4 protein, or the extracellular fragment of CTLA4, CTLA4ECD (SEQ ID NO: 2), or a fragment comprising CTLA4ECD; it also includes a fusion protein of CTLA4ECD, e.g., a fragment fused to the Fc protein fragment of a mouse IgG (mFc) (SEQ ID NO: 3). However, as understood by those skilled in the art, a mutation or variation (including, but not limited to, substitution, deletion and/or addition) may be naturally produced in or artificially introduced into the amino acid sequence of the CTLA4 protein, without affecting its biological functions. Therefore, in the present invention, the term “CTLA4 protein” should include all such sequences, including the sequence as set forth in SEQ ID NO: 2, as well as its native or artificial variants. Furthermore, when reference is made to a sequence fragment of the CTLA4 protein, it not only includes a sequence fragment of SEQ ID NO: 2, but also includes the corresponding sequence fragments of its native or artificial variants.

As used herein, unless specifically stated, B7 refers to B7-1 and/or B7-2; and their specific protein sequences refer to the sequences known in the art. Reference can be made to the sequences disclosed in the literatures of the prior art or GenBank, e.g., B7-1 (CD80, NCBI Gene ID: 941), B7-2 (CD86, NCBI Gene ID: 942).

As used herein, the term EC50 refers to concentration for 50% of maximal effect, i.e., the concentration causing 50% of the maximal effect.

As used herein, the term “antibody” refers to an immunoglobulin molecule which generally consists of two pairs of polypeptide chains (each pair has a “light” (L) chain and a “heavy” (H) chain). Antibody light chains can be classified as κ and λ light chain. Heavy chains can be classified as μ, δ, γ, α or ε, and the isotypes of antibody are defined as IgM, IgD, IgG, IgA and IgE, respectively. Within a light chain and heavy chain, a variable region and a constant region are joined via a “J” region of about 12 or more amino acids, and heavy chain further comprises a “D” region of about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of a CL domain. The constant region of antibody can mediate the binding of the immunoglobulin to host tissues or factors, including various cells of the immune system (e.g., effector cells) and the first component of the classical complement system (C1q). VH and VL regions can further be subdivided into regions having high variability (referred to as complementarity determining region (CDR)), interspersed with regions called framework regions (FR) which are relatively conserved. Each VH or VL consists of 3 CDRs and 4 FRs, arranged by the following order from the amino terminal to the carboxy terminal: FR1, CDR1, FR2, CDR2, FR3, CDR3, FR4. The variable regions (VH and VL) of each pair of heavy chain/light chain form an antibody binding site, respectively. The assignment of amino acids to each region or domain follows the definition provided in Kabat, Sequences of Proteins of Immunological Interest (National Institutes of Health, Bethesda, Md. (1987 and 1991)), or Chothia & Lesk (1987) J. Mol. Biol. 196: 901-917; Chothia et al. (1989) Nature 342: 878-883. The term “antibody” is not limited by any specific method for producing the antibody. For example, it includes, particularly, recombinant antibodies, monoclonal antibodies and polyclonal antibodies. Antibodies can be antibodies of different isotypes, e.g., IgG (e.g., IgG1, IgG2, IgG3 or IgG4 subtype), IgA1, IgA2, IgD, IgE or IgM antibodies.

As used herein, the term “antigen binding fragment” of antibody refers to a polypeptide comprising a fragment of a full-length antibody, which retains the ability to specifically bind to the antigen bound by the full-length antibody, and/or to compete with the full-length antibody for specifically binding to the antigen. It is also referred to as “antigen binding portion”. Generally, see Fundamental Immunology, Ch. 7 (Paul, W., ed., Second Edition, Raven Press, N.Y. (1989)), which is incorporated herein by reference in the entirety for all purposes. Antigen binding fragments of antibodies can be produced by recombinant DNA technique or enzymatic or chemical cleavage of intact antibodies. In some cases, antigen binding fragments include Fab, Fab′, F(ab′)2, Fd, Fv, dAb and complementarity determining region (CDR) fragment, single chain antibody (e.g., scFv), chimeric antibody, diabody and such polypeptides which comprises at least a portion of the antibody sufficient to confer the ability of specific antigen binding to the polypeptide.

As used herein, the term “Fd fragment” refers to an antibody fragment consisting of the VH and CH1 domains; the term “Fv fragment” refers to an antibody fragment consisting of the VL and VH domains of a single arm of antibody; the term “dAb fragment” refers to an antibody fragment consisting of the VH domain (Ward et al., Nature 341: 544-546 (1989)); the term “Fab fragment” refers to an antibody fragment consisting of the VL, VH, CL and CH1 domains; and the term “F(ab′)2 fragment” refers to an antibody fragment comprising two Fab fragments connected by disulfide bridges on the hinge region.

In some cases, the antigen binding fragment of antibody is a single chain antibody (e.g., scFv), wherein the VL and VH domains pair to each other via a linker which enables production of a single polypeptide chain to form a mono-valent molecule (see, e.g., Bird et al., Science 242: 423-426 (1988) and Huston et al., Proc. Natl. Acad. Sci. USA 85: 5879-5883 (1988)). Such scFv molecule can have the general structure: NH2-VL-Linker-VH-COOH or NH2-VH-Linker-VL-COOH. Suitable linkers from the prior art consist of the repeated GGGGS amino acid sequence or its variants. For example, a linker having the amino acid sequence (GGGGS)4 can be used, but its variants can also be used (Holliger et al. (1993) Proc. Natl. Acad. Sci. USA 90: 6444-6448). Other linkers useful in the present invention are described in Alfthan et al. (1995) Protein Eng. 8: 725-731; Choi et al. (2001) Eur. J. Immunol. 31: 94-106; Hu et al. (1996) Cancer Res. 56: 3055-3061; Kipriyanov et al. (1999) J. Mol. Biol. 293: 41-56 and Roovers et al. (2001) Cancer Immunol.

In some cases, the antigen binding fragment of antibody is a diabody (a bivalent antibody), wherein the VH and VL domains are expressed on a single polypeptide chain. However, the linker exploited is too short that the two domains on the same chain cannot pair with each other, and are forced to pair with the complemental domain on another chain. By this way, two antigen binding sites are formed (see, e.g., Holliger P. et al., Proc. Natl. Acad. Sci. USA 90: 6444-6448 (1993) and Poljak R. J. et al., Structure 2: 1121-1123 (1994)).

Antigen binding fragments of antibodies (e.g., the above antibody fragments) can be obtained from given antibodies (e.g., the monoclonal antibodies 4B3, 13A10, 12B9, or 4H4 provided in the present invention) using conventional technologies which are known to those skilled in the art (e.g., recombinant DNA technique or enzymatic or chemical cleavage method), and can be screened for specificity in the same way as that of intact antibodies.

Herein, unless explicitly indicted in the context, when reference is made to the term “antibody”, it not only includes intact antibodies, but also includes antigen binding fragments of antibodies.

As used herein, the terms “mAb” or “monoclonal antibody” refers to an antibody or antibody fragment from a population of highly homogenous antibody molecules, i.e., the individual antibodies comprising the population are identical except for possible naturally occurring mutations. Monoclonal antibodies are highly specific to a single epitope on the antigen. In contrast to monoclonal antibodies, polyclonal antibody preparations typically include at least two or more different antibodies recognizing different epitopes on the antigen. Monoclonal antibodies can generally be obtained using the hybridoma technique first described by Kohler et al. (Nature, 256: 495, 1975), or can be obtained using the recombinant DNA technique (see U.S. Pat. No. 4,816,567, for example).

As used herein, a monoclonal antibody mentioned with a number is identical with the monoclonal antibody obtained from a hybridoma mentioned with the same number. For example, the monoclonal antibody 4B3 (or 13A10, 12B9 or 4H4) is identical to the antibody obtained from the hybridoma cell line 4B3 (or 13A10, 12B9 or 4H4) or its subclones or descendent cells.

As used herein, the term “chimeric antibody” refers to such an antibody, in which a portion of the light chain and/or heavy chain is derived from an antibody (which can be derived from a particular species or belong to a particular antibody class or subclass), while another portion of the light chain and/or heavy chain is derived from another antibody (which can be derived from an identical or different species or belong to an identical or different antibody class or subclass), as long as it retains the activity to bind to the target antigen (U.S. Pat. No. 4,816,567 awarded to Cabilly et al.; Morrison et al., Proc. Natl. Acad. Sci. USA, 81: 6851-6855 (1984)).

As used herein, the term “humanized antibody” refers to the antibody or antibody fragment obtained after replacing all or some CDRs of a human immunoglobulin (recipient antibody) with CDRs of a non-human antibody (donor antibody), wherein the donor antibody can be a non-human (e.g., mouse, rat or rabbit) antibody having the desired specificity, affinity or reactivity. Furthermore, some amino acid residues of the framework regions (FRs) of the recipient antibody can be replaced with corresponding amino acid residues of the non-human antibody or amino acid residues of other antibodies so as to further improve or optimize the performance of the antibody. For more details about humanized antibodies, see, e.g., Jones et al., Nature, 321: 522-525 (1986); Reichmann et al., Nature, 332: 323-329 (1988); Presta, Curr. Op. Struct. Biol., 2: 593-596 (1992); and Clark, Immunol. Today 21: 397-402 (2000).

As used herein, a “neutralizing antibody” refers to an antibody or antibody fragment that can remove or significantly reduce the virulence of the target virus (e.g., the ability to infect cells).

As used herein, the term “epitope” refers to the prat on an antigen specifically bound by an immunoglobulin or antibody. In the art, “epitope” is also called “antigenic determinant”. An epitope or antigenic determinant generally consists of the chemically active surface groups of the molecule, e.g., amino acid or carbohydrate compounds or sugar side chains, and generally has specific three-dimensional structural characteristics and specific charge characteristics. For example, an epitope generally comprises at least 3, 4, 5, 6, 7, 8, 9, 10, 11, 12, 13, 14, or 15 consecutive or inconsecutive amino acids in a distinct spatial conformation. It can be a “linear” or “conformational” epitope. See, e.g., Epitope Mapping Protocols in Methods in Molecular Biology, Vol. 66, G. E. Morris, Ed. (1996). In a linear epitope, all the points of the interaction between the protein and the interacting molecule (e.g., antibody) are present along the primary amino acid sequence of the protein in a line. In a conformational epitope, the interacting points are present as spanning the amino acid residues of the protein which are separate from each other.

As used herein, the term “isolated” means being obtained from the native state by artificial means. If an “isolated” substance or component occurs in the nature, it is likely that its natural environment has changed, or the substance has been isolated from the natural environment, or both. For example, unisolated polynucleotides or polypeptides naturally occur in a living animal in vivo, and identical polynucleotides or polypeptides of high purity isolated from such natural state are described as “isolated”. The term “isolated” does not exclude mixture with artificial or synthetic substances, and does not exclude the presence of other impurities which do not affect the activities of the substance.

As used herein, the term “E. coli expression system” refers to an expression system consisting of E. coli (strain) and the vector, wherein E. coli (strain) is derived from strains commercially available, e.g., but not limited to GI698, ER2566, BL21(DE3), B834(DE3), and BLR(DE3).

As used herein, the term “vector” refers to a nucleic acid carrying tool into which a polynucleotide can be inserted. When a vector enables the expression of the protein encoded by the inserted polynucleotide, the vector is called expression vector. A vector can be introduced into a host cell by transformation, transduction or transfection, such that the genetic substance component carried by the vector is expressed in the host cell. Vectors are well known to those skilled in the art, including, but not limited to: plasmid; phagemid; cosmid; artificial chromosome, e.g., yeast artificial chromosome (YAC), bacterial artificial chromosome (BAC) or P1-derived artificial chromosome (PAC); bacteriophage, e.g., k phage or M13 phage as well as animal virus, and the like. Animal viruses which can be used as a vector, include, but not limited to, retrovirus (including lentivirus), adenovirus, adeno-associated virus, herpes virus (e.g., herpes simplex virus), poxvirus, baculovirus, papilloma virus, papova virus (e.g., SV40). A vector can comprise several components for controlling the expression, including, but not limited to, promoter sequence, transcription initiation sequence, enhancer sequence, selection component and reporter gene. Moreover, a vector can also comprise replication initiation site.

As used herein, the term “host cell” refers to cells which can be used for introduction of a vector, including, but not limited to, prokaryotic cells such as E. coli or Bacillus subtilis, fungal cells such as yeast cells or Aspergillus, insect cells such as S2 Drosophila cell or Sf9, or animal cells such as fibroblast, CHO cell, COS cell, NSO cell, HeLa cell, BHK cell, HEK 293 cell or human cell.

As used herein, the term “identity” is used to describe the sequence matching between two polypeptides or between two nucleic acids. When the corresponding positions in two sequences compared are occupied by the same base or amino acid monomer subunit (for example, the corresponding positions in two DNA molecules are both occupied by adenine, or the corresponding positions in two polypeptides are both occupied by lysine), the molecules are identical at that position. The “percent identity” between two sequences is a function of the number of the matching positions shared by these two sequences divided by the number of the positions compared×100. For example, if 6 among 10 positions of two sequences match, these two sequences have 60% identity. For example, DNA sequences CTGACT and CAGGTT share 50% identity (3 among 6 positions match in total). Generally, two sequences are compared after alignment to generate maximal identity. For example, such alignment can be conveniently achieved using a computer program, e.g., the Align program (DNAstar, Inc.), by the method of Needleman et al. (1970) J. Mol. Biol. 48: 443-453. Furthermore, the algorithm of E. Meyers and W. Miller (Comput. Appl. Biosci., 4: 11-17 (1988)) incorporated into the ALIGN program (version 2.0) can be used to determine the percent identity between two amino acid sequences, using the PAM120 weight residue table, a Gap length penalty of 12 and a gap penalty of 4. Moreover, the algorithm of Needleman and Wunsch (J Mol Biol. 48: 444-453 (1970)) incorporated into the GAP program of the GCG package (available at www.gcg.com) can be used to determine the percent identity between two amino acid sequences, using the Blossum 62 matrix or PAM250 matrix, a gap weight of 16, 14, 12, 10, 8, 6, or 4 and a length weight of 1, 2, 3, 4, 5, or 6.

As used herein, the term “conservative substitution” refers to amino acid substitutions that do not disadvantageously affect or alter the essential properties of the protein/polypeptide comprising the amino acid sequence. For example, conservative substitutions can be introduced by standard techniques known in the art, such as site-directed mutagenesis and PCR-mediated mutagenesis. Conservative amino acid substitutions include those wherein an amino acid residue is replaced with an amino acid residue having a similar side chain, e.g., a residue similar to the corresponding amino acid residue in terms of physics or function (e.g., having similar size, shape, charge, chemical properties, including the ability to form a covalent bond or hydrogen bond, or the like). Families of amino acid residues having similar side chains have been defined in the art. These families include amino acids having a basic side chain (e.g., lysine, arginine and histidine), an acidic side chain (e.g., aspartic acid and glutamic acid), an uncharged polar side chain (e.g., glycine, asparagine, glutamine, serine, threonine, tyrosine, cysteine, and tryptophan), a non-polar side chain (e.g., alanine, valine, leucine, isoleucine, proline, phenylalanine, and methionine), a beta-branched side chain (e.g., threonine, valine and isoleucine), and an aromatic side chain (e.g., tyrosine, phenylalanine, tryptophan, and histidine). Thus, it is preferred to replace the corresponding amino acid residue with another amino acid residue from the same side chain family. Methods of identifying amino acid conservative substitutions are well known in the art (see, e.g., Brummell et al., Biochem., 32: 1180-1187 (1993); Kobayashi et al., Protein Eng. 12(10): 879-884 (1999); and Burks et al., Proc. Natl. Acad. Sci. USA, 94: 412-417 (1997), which are incorporated herein by reference).

As used herein, the term “immunogenicity” refers to the ability to stimulate the body to generate specific antibodies or sensitize lymphocytes. It not only refers to the properties of the antigen which can stimulate specific immune cells so as to induce activation, proliferation and differentiation of the immune cells, and ultimately production of immune effector substances such as antibodies, and can sensitize lymphocytes, but also refers to the specific immune responses by the body's immune system to produce antibodies or sensitize T lymphocytes after the stimulation of the body by the antigen. Immunogenicity is the most important property of the antigen. Whether an antigen can successfully induce immune response in a host depends on three factors: the nature of the antigen, the reactivity of the host and the manner of immunization.

As used herein, the term “specific binding” refers to the non-random binding reaction between two molecules, such as the reaction between an antibody and its targeted antigen. In some embodiments, an antibody specifically binding an antigen (or an antibody specific for an antigen) means the antibody binds the antigen with an affinity (KD) less than about 10⁻⁵ M, e.g., less than about 10⁻⁶ M, 10⁻⁷ M, 10⁻⁸ M, 10⁻⁹ M, or 10⁻¹⁰ M or less.

As used herein, the term “KD” refers to the dissociation equilibration constant of a particular antibody-antigen interaction, which is used to describe the binding affinity between the antibody and the antigen. The less the equilibration dissociation constant is, the tighter the antibody-antigen binding is and the higher the affinity between the antibody and antigen is. Generally, an antibody (e.g., the monoclonal antibodies 4B3, 13A10, 12B9, or 4H4 of the present invention) binds the antigen (e.g., the L1 protein) with a dissociation equilibration constant (KD) less than about 10⁻⁵ M, e.g., less than about 10⁻⁶ M, 10⁻⁷ M, 10⁻⁸ M, 10⁻⁹ M, or 10⁻¹⁰ M or less, e.g., as determined using Surface Plasmon Resonance (SPR) on a BIACORE instrument.

As used herein, the terms “monoclonal antibody” and “mAb” have the same meaning, and can be used interchangeably. Also, the terms “polyclonal antibody” and “pAb” have the same meaning, and can be used interchangeably. Again, the terms “polypeptide” and “protein” have the same meaning, and can be used interchangeably. Furthermore, in the present invention, amino acids are generally represented by single-letter or three-letter abbreviations well known in the art. For example, alanine can be represented by A or Ala.

As used herein, the terms “hybridoma” and “hybridoma cell line” can be used interchangeably. Moreover, when reference is made to the term “hybridoma” or “hybridoma cell line”, it also comprises the subclones and descendent cells of the hybridoma. For example, when reference is made to the hybridoma cell line 4B3, it also comprises the subclones and descendent cells of the hybridoma cell line 4B3.

As used herein, the term “pharmaceutically acceptable vector and/or excipient” refers to vector and/or excipient compatible to the subject and the active component in pharmacology and/or physiology, which are well known in the art (see, e.g., Remington's Pharmaceutical Sciences. Edited by Gennaro A R, 19th ed. Pennsylvania: Mack Publishing Company, 1995), and include but not limited to: pH adjusting agent, surfactant, adjuvant, ionic intensity enhancer. For example, pH adjusting agent includes but not limited to phosphate buffer; surfactant includes but not limited to cationic, anionic or nonionic surfactant, e.g., Tween-80; and ionic intensity enhancer includes but not limited to sodium chloride.

As used herein, the term “adjuvant” refers to non-specific immune enhancer, which can enhance the immune response of the body to the antigen or change the type of the immune response when delivered together with an antigen or in advance into the body. There are many adjuvants, including but not limited to aluminum adjuvant (e.g., aluminum hydroxide), Freund's adjuvant (e.g., complete Freund's adjuvant and incomplete Freund's adjuvant), Corynebacterium parvum, lipopolysaccharide, cytokine, and the like. Freund's adjuvant is the most commonly used one in animal trials at present, and aluminum hydroxide is the widely used one in clinical experiments.

As used herein, the term “effective amount” refers to an amount sufficient to achieve or at least partially achieve the desired effects. For example, prophylactically effective amount for a disease (e.g., a disease associated with excessive binding of CTLA4 to B7 or CTLA4 activity such as tumor) refers to an amount sufficient to prevent, arrest, or delay the development of a disease (e.g., a disease associated with excessive binding of CTLA4 to B7 or CTLA4 activity such as tumor); and therapeutically effective amount for a disease refers to an amount sufficient to cure or at least partially arrest a disease and its complications in a patient suffering from the disease. It is well within the skills of those skilled in the art to determine such effective amount. For example, a therapeutically effective amount will depend on the severity of the disease to be treated, the general status of the immune system of the patient, the general status of the patient, e.g., age, body weight and sex, administration mode of the agent, other therapies administrated simultaneously, and the like.

### Beneficial Effects of the Invention

The monoclonal antibody 8D2 and its humanized antibodies of the present invention can specifically bind to CTLA4 very well. Among them, the antibodies 8D2 and 8D2(Re) bind to the murine CTLA4 antigen at a binding efficiency better than the control antibodies 10D1 (Alan J. Korman, Edward L. Halk, et al., HUMAN CTLA-4 ANTIBODIES, U.S. Pat. No. 6,984,720 B1) and 11.2.1 (Douglas Charles Hanson, Mark Joseph Neveu, et al., Human monoclonal antibodies to CTLA-4, U.S. Pat. No. 682,736 B1). The humanized antibody 8D2H1L1 binds to the murine CTLA4 antigen at a binding efficiency better than the control antibody 10D1, and comparable to 11.2.1. The humanized antibody 8D2H2L2 binds to the human CTLA4 antigen at a binding efficiency comparable to 10D1. The humanized antibodies 8D2H2L2 and 8D2H3L3 bind to the monkey CTLA4 antigen at a binding efficiency comparable to 10D1. The antibodies 8D2H2L15 and 8D2H2L17 bind to the human CTLA4 antigen at a binding efficiency better than the control antibodies 10D1 and 11.2.1. The antibodies 8D2, 8D2(Re), and the 8D2 humanized antibodies 8D2H1L1, 8D2H2L2, 8D2H3L3, 8D2H2L15, and 8D2H2L17 can compete with B7 for binding to the antigen CTLA4. Among them, 8D2, 8D2(Re), 8D2H1L1, and 8D2H2L2 are stronger than 10D1 in competing with B7-2 for binding to CTLA4; and 8D2H1L1, 8D2H2L2, 8D2H3L3, 8D2H2L15, and 8D2H2L17 are all stronger than the antibodies 10D1 and 11.2.1 in competing with B7-1 and B7-2 for binding to CTLA4.

The monoclonal antibody 8D2 and its humanized antibodies of the present invention can block the binding of CLTA4 to B7, specifically relieve the immunosuppression on the body by CTLA4, and activate T lymphocytes very effectively. Among them, 8D2H2L2 and 8D2H2L15 are stronger than the control antibodies 10D1 and 11.2.1 in activating T lymphocytes.

### BRIEF DESCRIPTION OF THE DRAWINGS

FIG. 1. Results of SDS-PAGE of the CTLA4ECD-mFc fusion protein. The samples and their loading amounts in the 4 lanes, from left to right, were: M, Marker, 10 μL; CTLA4ECD-mFc fusion protein, 1 μg; CTLA4ECD-mFc fusion protein, 2 μg; CTLA4ECD-mFc fusion protein, 3 μg.

FIG. 2. Results of SDS-PAGE of the 8D2 antibody. The samples and their loading amounts in the 4 lanes, from left to right, were: M, marker, 10 μL; an antibody sample in the reductive loading buffer for protein electrophoresis, 0.3 μg; the non-reductive loading buffer for protein electrophoresis, 2 μL; an antibody sample in the non-reductive loading buffer for protein electrophoresis, 0.3 μg.

FIG. 3. Results of SDS-PAGE of the 8D2 recombinant antibody (8D2(Re)). The samples and their loading amounts in the 4 lanes, from left to right, were: M, marker, 10 μL; an antibody sample in the reductive loading buffer for protein electrophoresis, 1 μg; the non-reductive loading buffer for protein electrophoresis, 2 μL; an antibody sample in the non-reductive loading buffer for protein electrophoresis, 1 μg.

FIG. 4. Results of SDS-PAGE of the humanized antibody of 8D2, 8D2H1L1. The samples and their loading amounts in the 4 lanes, from left to right, were: M, marker, 10 μL; lane 1, an antibody sample in the reductive loading buffer for protein electrophoresis, 1 μg; lane 2, the non-reductive loading buffer for protein electrophoresis, 2 μL; lane 3, an antibody sample in the non-reductive loading buffer for protein electrophoresis, 1 μg.

FIG. 5. Results of SDS-PAGE of the humanized antibody of 8D2, 8D2H2L2. The samples and their loading amounts in the 4 lanes, from left to right, were: M, marker, 10 μL; lane 1, an antibody sample in the reductive loading buffer for protein electrophoresis, 1 μg; lane 2, the non-reductive loading buffer for protein electrophoresis, 2 μL; lane 3, an antibody sample in the non-reductive loading buffer for protein electrophoresis, 1 μg.

FIG. 6. Results of SDS-PAGE of the humanized antibody of 8D2, 8D2H3L3. The samples and their loading amounts in the 4 lanes, from left to right, were: M, marker, 10 μL; lane 1, an antibody sample in the reductive loading buffer for protein electrophoresis, 1 μg; lane 2, the non-reductive loading buffer for protein electrophoresis, 2 μL; lane 3, an antibody sample in the non-reductive loading buffer for protein electrophoresis, 1 μg.

FIG. 7. Results of SDS-PAGE of the humanized antibody of 8D2, 8D2H2L15. The samples and their loading amounts were: M, marker, 10 μL; lane 2, an antibody sample in the non-reductive loading buffer for protein electrophoresis, 1 μg; lane 1, an antibody sample in the reductive loading buffer for protein electrophoresis, 1 μg.

FIG. 8. Results of SDS-PAGE of the humanized antibody of 8D2, 8D2H2L17. The samples and their loading amounts were: M, marker, 10 μL; lane 2, an antibody sample in the non-reductive loading buffer for protein electrophoresis, 1 μg; lane 1, an antibody sample in the reductive loading buffer for protein electrophoresis, 1 μg.

FIG. 9. A histogram showing the expression of CTLA4 on non-labeled 293F cells, isotype control, and 293F-CTLA4 cells as detected using a flow cytometry (cell number—fluorescence (FITC)).

FIG. 10. Mean fluorescence intensity (MFI) of the expression of CTLA4 on non-labeled 293F cells, isotype control, and 293F-CTLA4 cells as detected using a flow cytometry.

FIG. 11. The EC50 results of the binding of the mAb 8D2 to the labeled 293F-CTLA4 cells.

FIG. 12. The EC50 results of the binding of the 8D2(Re) antibody to the labeled 293F-CTLA4 cells.

FIG. 13. The EC50 results of the binding of 8D2H1L1 to the labeled 293F-CTLA4 cells.

FIG. 14. The EC50 results of the binding of 8D2H2L2 to the labeled 293F-CTLA4 cells.

FIG. 15. The EC50 results of the binding of 8D2H3L3 to the labeled 293F-CTLA4 cells.

FIG. 16. Determination of the binding of the antibodies 8D2, 8D2H1L1, and 8D2(Re) and control antibodies 10D1 and 11.2.1 to CTLA4 using the ELISA method.

FIG. 17. Determination of the binding of the recombinant antibodies 8D2H2L2 and 8D2H3L3 and control antibodies 10D1 and 11.2.1 to human CTLA4 using the ELISA method.

FIG. 18. Determination of the binding of the recombinant antibodies 8D2H2L2 and 8D2H3L3 and control antibodies 10D1 and 11.2.1 to monkey CTLA4 using the ELISA method.

FIG. 19. Determination of the binding of the recombinant antibodies 8D2H2L2(20150327), 8D2H2L15, 8D2H2L17, and 8D2H2L2 (20140422) and control antibodies 10D1 and 11.2.1 to monkey CTLA4 using the ELISA method.

FIG. 20. Results of ELISA for competition of the antibodies 8D2H1L1 and control antibodies 10D1 and 11.2.1 with B7-1.

FIG. 21. Results of ELISA for competition of the antibodies 8D2, 8D2H1L1, and 8D2(Re), and control antibodies 10D1 and 11.2.1 with B7-2.

FIG. 22. Results of ELISA for competition of the 8D2H2L2 and 8D2H3L3 antibodies and control antibodies 10D1 and 11.2.1 with B7-1.

FIG. 23. Results of ELISA for competition of the 8D2H2L2 and 8D2H3L3 antibodies and control antibodies 10D1 and 11.2.1 with B7-2.

FIG. 24. Results of ELISA for competition of the 8D2H2L2(20150327), 8D2H2L15, 8D2H2L17, 8D2H2L2(20140422) antibodies and control antibodies 10D1 and 11.2.1 with B7-1.

FIG. 25. Results of ELISA for competition of the 8D2H2L2(20150327), 8D2H2L15, 8D2H2L17, 8D2H2L2(20140422) antibodies and control antibodies 10D1 and 11.2.1 with B7-2.

FIG. 26. Effects on the level of IL-2 secretion by the T lymphocytes as detected by the ELISA method after co-culture of 72 hours with peripheral blood mononuclear cells (PBMC), Raji cells and the humanized antibodies 8D2H1L1, 8D2H2L2, or 8D2H3L3, respectively. The results show that the humanized antibodies of the mAb 8D2 increased the IL-2 secretion by the T lymphocytes by preventing the receptor of CTLA4.

FIG. 27. Effects on the level of IL-2 secretion by the T lymphocytes as detected by the ELISA method after co-culture of 72 hours with peripheral blood mononuclear cells (PBMC), Raji cells and the humanized antibodies 8D2H2L2, 8D2H2L15, or 8D2H2L17 and control antibodies 10D1 and 11.2.1, respectively. The results show that the humanized antibodies of the mAb 8D2 increased the IL-2 secretion by the T lymphocytes by preventing the receptor of CTLA4.

FIG. 28. The growth curve of the tumor subcutaneously transplanted in the hu-SCID-Raji model treated with 8D2H2L2.

### SPECIFIC EMBODIMENTS

The embodiments of the invention will be described below in details with reference to the Examples. Those skilled in the art will understand that the following Examples are provided merely to illustrate the invention. They should not be construed, whatsoever, as to limit the scope of the invention. Examples, for which specific techniques or conditions are not described, were performed using the techniques or conditions disclosed in the literatures of the art (e.g., written by J. Sambrook et al., translated by Peitang HUANG et al., Molecular Cloning: A Laboratory Manual, Third Edition, Science Press) or following the instructions provided with the products. Reagents and instruments, for which the supplier is not indicated, are conventional products which are commercially available.

In the following Examples of the invention, the BALB/C mice were purchased from Guangdong Medical Laboratory Animal Center.

In the following Examples of the invention, the T cells used were obtained from Akeso Biopharma Inc., Zhongshan.

The control antibody, 10D1, was prepared according to U.S. Pat. No. 6,984,720 B1; and 11.2.1 according to U.S. Pat. No. 6,682,736 B1.

### Example 1. Generation of the CTLA4-8D2 Hybridoma Cell Line LT001 and Preparation of the Monoclonal Antibody 8D2

Recombinant CTLA4 was expressed in a mammalian cell expression system for immunizing mice as antigen, and hybridoma cells were obtained by fusing mouse spleen cells with myeloma cells. A hybridoma cell line (the CTLA4-8D2 hybridoma cell line LT001) was obtained after screening a great number of samples. Said cell line could secret the monoclonal antibody 8D2, which specifically binds CTLA4. The specific methods are described below.

1. Synthesis of the CTLA4ECD-mFc gene

According to the design (SEQ ID NO: 3), the amino acid sequence (SEQ ID NO: 2) corresponding to the extracellular fragment of the CTLA4 gene (Cytotoxic T-Lymphocyte Antigen 4, NCBI Gene ID: 1493, SEQ ID NO: 1) (CTLA4ECD) was fused to the Fc protein fragment of mouse IgG (mFc), wherein mFc refers to the Fc protein fragment of mouse IgG with the amino acid sequence as shown by the underlined part of SEQ ID NO: 3.

In order to increase the expression efficiency of the gene of interest in the 293f cell expression system, the nucleic acid sequence encoding the SEQ ID NO: 3 protein sequence was optimized at Genscript Co., mainly taking the factors such as codon preference, GC content, secondary structures of mRNA, and repeated sequences into consideration. The final optimized gene encoding the CTLA4ECD-mFc fusion protein has the following sequence (SEQ ID NO: 4), and was synthesized at Genscript Co.

The sequence of the CTLA4ECD gene (375 bp):

(SEQ ID NO: 1)
GCAATGCACGTGGCCCAGCCTGCTGTGGTACTGGCCAGCAG
CCGAGGCATCGCCAGCTTTGTGTGTGAGTATGCATCTCCAGGCA
AAGCCACTGAGGTCCGGGTGACAGTGCTTCGGCAGGCTGACAG
CCAGGTGACTGAAGTCTGTGCGGCAACCTACATGATGGGGAAT
GAGTTGACCTTCCTAGATGATTCCATCTGCACGGGCACCTCCAG
TGGAAATCAAGTGAACCTCACTATCCAAGGACTGAGGGCCATGG
ACACGGGACTCTACATCTGCAAGGTGGAGCTCATGTACCCACCG
CCATACTACCTGGGCATAGGCAACGGAACCCAGATTTATGTAAT
TGATCCAGAACCGTGCCCAGATTCTGAC

The sequence of the protein encoded by CTLA4ECD (125 aa):

(SEQ ID NO: 2)
AMHVAQPAVVLASSRGIASFVCEYASPGKATEVRVTVLRQADS
QVTEVCAATYMMGNELTFLDDSICTGTSSGNQVNLTIQGLRAMDT
GLYICKVELMYPPPYYLGIGNGTQIYVIDPEPCPDSD

The sequence of the CTLA4ECD-mFc fusion protein (364 aa):
wherein the CTLA4ECD portion is underlined with a waving line, and the mFc portion is underlined with a solid line.

(SEQ ID NO: 3)

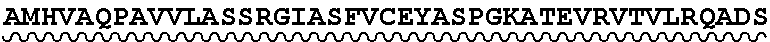

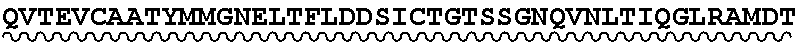

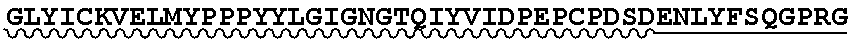

PTIKPCPPCKCPAPNLLGGPSVFIFPPKIKDVLMISLSPIVTCVVVDVS
EDDPDVOISWFVNNVEVHTAQTOTHREDYNSTLRVVSALPIQHOD
WMSGKEFKCKVNNKDLPAPIERTISKPKGSVRAPQVYVLPPPEEEM
TKKQVTLTCMVTDFMPEDIYVEWTNNGKTELNYKNTEPVLDSDGS
YFMYSKLRVEKKNWVERNSYSCSVVHEGLHNHHTTKSFSRTPGK

The coding sequence of the gene corresponding to the CTLA4ECD-mFc fusion protein (1092 bp):
wherein the CTLA4ECD portion is underlined with a waving line, and the mFc portion is underlined with a solid line.

(SEQ ID NO: 4)

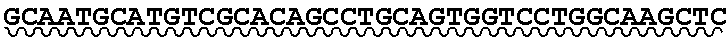

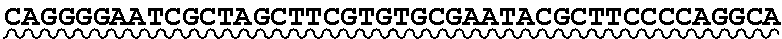

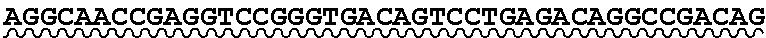

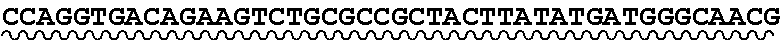

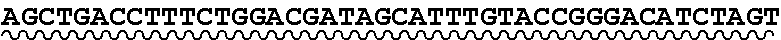

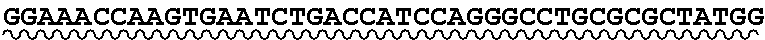

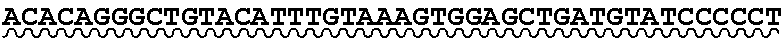

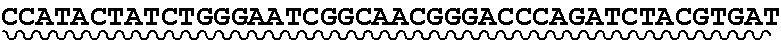

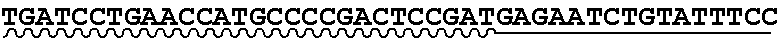

AGGGACCACGAGGCCCCACAATTAAGCCATGTCCCCCTTGCAAA
TGTCCTGCACCAAACCTGCTGGGAGGACCAAGCGTGTTCATCTT
TCCACCCAAGATCAAGGACGTGCTGATGATCTCACTGAGCCCCA
TTGTGACCTGCGTGGTCGTGGACGTGAGCGAGGACGATCCTGA
TGTGCAGATCAGTTGGTTCGTCAACAATGTGGAAGTCCACACAG
CTCAGACTCAGACCCATAGGGAGGATTACAATAGTACTCTGCGC
GTCGTGTCAGCACTGCCCATTCAGCACCAGGACTGGATGAGCG
GCAAGGAGTTCAAGTGCAAAGTGAACAACAAGGATCTGCCCGC
ACCTATCGAGAGAACTATTTCCAAGCCTAAAGGGTCTGTGAGGG
CCCCACAGGTGTATGTCCTGCCTCCACCCGAGGAAGAGATGACT
AAGAAACAGGTGACACTGACTTGTATGGTCACCGACTTCATGCC
CGAAGATATCTACGTGGAGTGGACTAACAATGGGAAGACCGAA
CTGAACTATAAAAATACAGAGCCTGTGCTGGACTCAGATGGAAG
CTACTTTATGTATAGCAAGCTGCGAGTGGAAAAGAAAAACTGGG
TCGAGCGGAACAGCTACTCTTGTAGTGTGGTCCACGAAGGGCTG
CATAATCACCACACCACTAAATCATTCTCCCGAACTCCAGGCAA
A

2. Generation of the pUC57Simple-CTLA4ECD-mFc plasmid

The synthesized CTLA4ECD-mFc fusion gene (SEQ ID NO: 4) was cloned into the pUC57simple expression vector (provided by Genscript Co.) at Genscript Co., resulting in the pUC57simple-CTLA4ECD-mFc plasmid.

3. Construction of the pcDNA3.1-CTLA4ECD-mFc recombinant plasmid

The pUC57simple-CTLA4ECD-mFc plasmid was digested with the endonucleases Xba I and BamH I. The CTLA4ECD-mFc fusion gene fragment was recovered via electrophoresis and was ligated into the pcDNA3.1 expression vector (purchased from Invitrogen Co.). The resultant pcDNA3.1-CTLA4ECD-mFc plasmid was used to transfect the competent cells of the DH5a strain of E. coli (purchased from TIANGEN Co.). Transfection and culture were performed following the instructions. E. coli colonies positive for pcDNA3.1-CTLA4ECD-mFc were screened out and propagated following conventional methods. Then, the pcDNA3.1-CTLA4ECD-mFc recombinant plasmid was extracted using a kit (purchased from Tiangen Biotech (Beijing) Co. LTD, DP103-03) following the instructions provided with the kit.

4. Cells of 293F (purchased from Invitrogen Co.) were transfected with the pcDNA3.1-CTLA4ECD-mFc recombinant plasmid using the lipofectamin transfection kit (purchased from Invitrogen Co.).

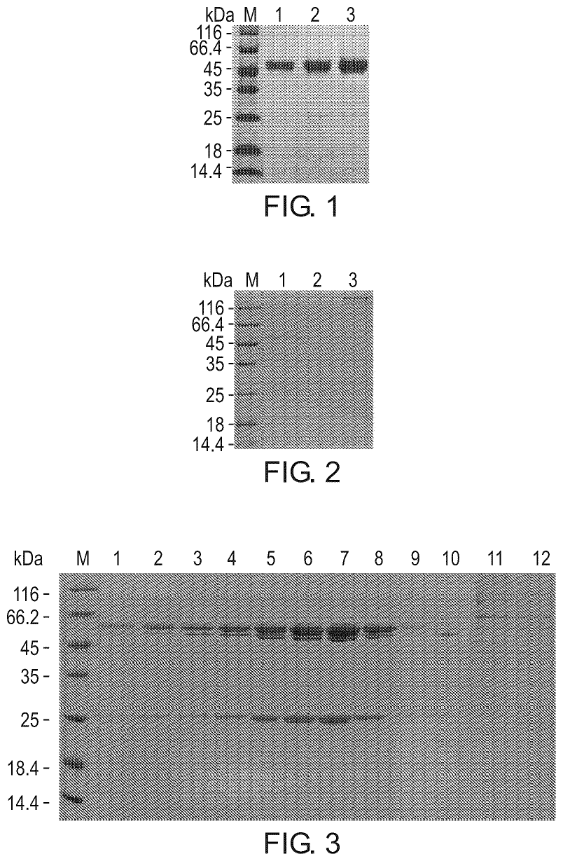

5. Seven days after transfecting 293F cells with the pcDNA3.1-CTLA4ECD-mFc recombinant plasmid, the CTLA4ECD-mFc fusion protein was purified from the culture liquid by high-speed centrifugation, vacuum filtration through a microporous filter membrane, and HiTrap protein A HP column chromatography. After purification, samples were taken, added into the reductive loading buffer for protein electrophoresis, and examined by SDS-PAGE electrophoresis. As shown in FIG. 1, the protein of interest is shown as a band at about 45 kD.

6. Generation of the CTLA4-8D2 hybridoma cell line LT001

Using the CTLA4ECD-mFc fusion protein as the antigen, hybridoma cells were obtained by fusing the splenic cells from the immunized BALB/C mice (purchased from Guangdong Medical Laboratory Animal Center) with mouse myeloma cells following an established method (e.g., Stewart, S. J., “Monoclonal Antibody Production”, in Basic Methods in antibody Production and Characterization, Eds. G. C. Howard and D. R. Bethell, Boca Raton: CRC Press, 2000).

CTLA4 was used as the antigen to coat an ELISA plate, and hybridoma cells secreting novel antibodies specifically binding to CTLA4 were obtained by an indirect ELISA screening. Hybridoma cell lines secreting monoclonal antibodies which competed with the ligand B7-1 (CD80, NCBI Gene ID: 941) or B7-2 (CD86, NCBI Gene ID: 942) for binding to CTLA4 were obtained by a competitive ELISA screening from the hybridoma cells obtained in the indirect ELISA screening. A stable hybridoma cell line was obtained via limited dilution. The hybridoma cell line was designated as the CTLA4-8D2 hybridoma cell line, and the CTLA4-8D2 stable cell line was obtained via limited dilution (also referred to as LT001 in the present invention; the monoclonal antibody secreted by it was designated as 8D2).

7. Preparation of the antibody 8G2

The CTLA4-8D2 (LT001) cell line of the present invention was cultured in a medium supplemented with 10% fetal bovine serum with low IgG. Seven days later, the supernatant of the cell culture was collected to purify the antibody 8D2.

8. Detection of the 8D2 antibody by SDS-PAGE

Purified samples were added into the reductive loading buffer for protein electrophoresis and the non-reductive loading buffer for protein electrophoresis. After boiling, detection was performed. The results show that the protein of interest is shown as two band at about 50 kD and 25 kD for the reductive protein sample, or as a band at about 150 kD for the non-reductive protein sample (FIG. 2).

### Example 2. Determination of the Light Chain and Heavy Chain Sequences of the Monoclonal Antibody 8D2

Following the instructions of the Cultured Cell/Bacteria Total RNA Extraction Kit (Tiangen, Cat. No. DP430), mRNA was extracted from the CTLA4-8D2 hybridoma cell line (LT001 cell) generated in Example 1.

Following the instructions of the Invitrogen SuperScript® III First-Strand Synthesis System for RT-PCR kit, cDNA was synthesized and amplified by PCR. The PCR amplification product was immediately subjected to TA cloning, following the instructions of the pEASY-T1 Cloning Kit (TransGen, Cat. No. CT101). The product of TA cloning was immediately subjected to sequencing, and the sequencing results are provided below.

The results of DNA sequencing of the heavy chain variable region (345 bp):

(SEQ ID NO: 5)
GAGGTGAAACTGGACGAAACTGGCGGGGGGCTGGTGCAGC
CCGGACGACCTATGAAGCTGTCATGCGTCGCCAGCGGCTTCACC
TTTAGCGACAACTGGATGAATTGGGTGAGGCAGAGCCCAGAGA
AGGGGCTGGAATGGCTGGCTCAGATCCGCAACAAACCCTACAAT
TATGAGACCTACTATTCTGACAGTGTGAAGGGCCGGTTCACAAT
TTCCAGAGACGATTCTAAAAGCTCCGTCTACCTGCAGATGAACA
ATCTGAGAGGCGAAGATATGGGGATCTACTATTGCACAGCACAG
TTCGCTTATTGGGGACAGGGCACTCTGGTCACAGTCTCCGCC

The protein sequence encoded by it (115 aa):

(SEQ ID NO: 6)
EVKLDETGGGLVQPGRPMKLSCVASGFTFSDNWMNWVRQSP
EKGLEWLAQIRNKPYNYETYYSDSVKGRFTISRDDSKSSVY
LQMNNLRGEDMGIYYCTAQFAYWGQGTLVTVSA

The results of DNA sequencing of the light chain variable region (318 bp):

(SEQ ID NO: 7)
GACATTCAGATGACACAGAGTCCTGCTTCCCTGAGTGCCTC
AGTGGGGGAGACCGTCACAATCACTTGCGGCACCTCTGAAAACA
TCTACGGCGGGCTGAATTGGTATCAGCGGAAGCAGGGCAAAAG
TCCCCAGCTGCTGATCTTCGGAGCAACAAACCTGGCCGACGGCA
TGAGCTCCCGGTTTAGCGGGTCCGGATCTGGCAGACAGTACAG
CCTGAAGATTTCTAGTCTGCACCCAGACGATGTGGCTACTTACT
ATTGCCAGAATGTCCTGAGGAGTCCCTTCACCTTTGGGTCAGGA
ACAAAGCTGGAGATC

The protein sequence encoded by it (106 aa):

(SEQ ID NO: 8)
DIQMTQSPASLSASVGETVTITCGTSENIYGGLNWYQRKQGKS
PQLLIFGATNLADGMSSRFSGSGSGRQYSLKISSLHPDDVATYYCQN
VLRSPFTFGSGTKLEI

### Example 3. Design of the Light Chain and Heavy Chain Sequences of the Humanized Antibodies 8D2H1L1, 8D2H2L2, 8D2H3L3, 8D2H2L15 and 8D2H2L17

Based on the three-dimensional crystal structure of the CTLA4 protein (Nat. Struct. Biol. (1997) 4, p. 527) and the sequences of the 8D2 antibody obtained in Example 2, the structure of the antibody was modeled on computer. The variable region sequences of the antibodies 8D2H1L1, 8D2H2L2, 8D2H3L3, 8D2H2L15 and 8D2H2L17 were designed based on the antibody sequences and the structural model (the constant region sequences of the antibodies were from the NCBI database). The variable region sequences are provided below.

### 1. The Light Chain and Heavy Chain Sequences of the Monoclonal Antibody 8D2H1L1

The DNA sequence of the heavy chain variable region (345 bp):

(SEQ ID NO: 9)
GAAGTGCAGCTGGTCGAGTCCGGGGGGGGCCTGGTGCAGC
CAGGAGGATCAATGCGACTGAGCTGCGCCGCTTCCGGCTTCACC
TTCAGCGACAACTGGATGAATTGGGTCAGGCAGGCACCAGGAA
AGGGACTGGAGTGGCTGGCACAGATCCGCAACAAACCTTACAA
CTACGAAACTTACTACAGCGACTCCGTGAAGGGGCGGTTCACCA
TTTCTAGAGACGATTCTAAAAACAGTGTGTACCTGCAGATGAAT
AGCCTGAAGACCGAGGATACAGGAGTCTACTATTGTACCGCACA
GTTTGCTTATTGGGGGCAGGGCACTCTGGTGACAGTCTCTTCA

The protein sequence encoded by it (115 aa):

(SEQ ID NO: 10)
EVQLVESGGGLVQPGGSMRLSCAASGFTFSDNWMNWVRQAP
GKGLEWLAQIRNKPYNYETYYSDSVKGRFTISRDDSKNSVYLQMNS
LKTEDTGVYVCTAQFAYWGQGTLVTVSS

The DNA sequence of the light chain variable region (321 bp):

(SEQ ID NO: 11)
GACATTCAGATGACTCAGAGCCCTTCAAGCCTGTCCGCATC
TGTGGGCGACCGAGTCACCATCACATGCAGAACCTCCGAGAACA
TCTACGGCGGGCTGAATTGGTATCAGCGAAAGCAGGGGAAAAG
TCCCAAGCTGCTGATCTACGGGGCAACAAACCTGGCCAGCGGA
ATGAGCTCCAGATTCAGTGGATCAGGCAGCGGGACAGATTATAC
TCTGAAAATTTCTAGTCTGCACCCAGACGATGTGGCAACCTACT
ATTGCCAGAATGTCCTGAGGTCACCCTTCACCTTTGGAAGCGGC
ACAAAACTGGAGATCAAG

The protein sequence encoded by it (107 aa):

(SEQ ID NO: 12)
DIQMTQSPSSLSASVGDRVTITCRTSENIGGLNWYQRKQGKS
PKLLIYGATNLASGMSSRFSGSGSGTDYTLKISSLHPDDVATYYCQN
VLRSPFTFGSGTKLEIK

### 2. The Light Chain and Heavy Chain Sequences of the 8D2 Humanized Monoclonal Antibody 8D2H2L2

The DNA sequence of the heavy chain variable region (345 bp):

(SEQ ID NO: 13)
GAAGTGCAGCTGGTCGAGTCCGGGGGGGGCCTGGTGCAGC
CAGGAGGATCAATGCGACTGAGCTGCGCCGCTTCCGGCTTCACC
TTCAGCGACAACTGGATGAATTGGGTCAGGCAGGCACCAGGAA
AGGGACTGGAGTGGCTGGCACAGATCCGCAACAAACCTTACAA
CTACGAAACTTACTACAGCGCCTCCGTGAAGGGGCGGTTCACCA
TTTCTAGAGACGATTCTAAAAACAGTGTGTACCTGCAGATGAAT
AGCCTGAAGACCGAGGATACAGGAGTCTACTATTGTACCGCACA
GTTTGCTTATTGGGGGCAGGGCACTCTGGTGACAGTCTCTTCA

The DNA sequence of the light chain variable region (321 bp):

(SEQ ID NO: 14 )
EVQLVESGGGLVQPGGSMRLSCAASGFTFSDNWMNWVRQAP
GKGLEWLAQIRNKPYNYETYYSASVKGRFTISRDDSKNSVYLQMNS
LKTEDTGVYYCTAQFAYWGQGTLVTVSS

The protein sequence encoded by it (107 aa):

(SEQ ID NO: 15)
GACATTCAGATGACTCAGAGCCCTTCAAGCCTGAGTGCCTC
AGTGGGAGACCGGGTCACCATCACATGCAGAACCAGCGAGAAC
ATCTACGGCGGCCTGAACTGGTATCAGCGAAAGCCAGGCAAGA
GCCCCAAGCTGCTGATCTACGGGGCAACCAACCTGGCCTCTGGA
GTGAGCTCCAGATTCAGCGGCAGCGGCTCTGGGACCGACTATA
CTCTGACCATTTCTAGTCTGCAGCCTGAAGATGTGGCAACATAC
TATTGCCAGAATGTCCTGAGGTCCCCATTCACCTTTGGATCTGG
CACCAAGCTGGAGATCAAG

(SEQ ID NO: 16)
DIQMTQSPSSLSASVGDRVTITCRTSENIYGGLNWYQRKPGKSP
KLLIYGATNLASGVSSRFSGSGSGTDYTLTISSLQPEDVATYYCQNV
LRSPFTFGSGTKLEIK

### 3. The Light Chain and Heavy Chain Sequences of the 8D2 Humanized Monoclonal Antibody 8D2H3L3

The DNA sequence of the heavy chain variable region (345 bp):

(SEQ ID NO: 17)
GAGGTGCAGCTGGTCGAGTCTGGAGGCGGCCTGGTGCAGC
CCGGCGGGTCACTGCGACTGAGCTGCGCCGCTTCCGGCTTCAC
CTTCAGCGACAACTGGATGAATTGGGTGAGGCAGGCACCCGGG
AAGGGGCTGGAGTGGGTCGCTCAGATCCGCAACAAACCTTACA
ATTATGAGACAGAATACGCAGCCTCTGTGAAGGGGCGGTTCACT
ATTAGTAGAGACGATAGCAAGAACAGCGCCTATCTGCAGATGAA
TAGCCTGAAGACCGAAGATACAGCCGTCTACTATTGTACAGCTC
AGTTTGCATACTGGGGCCAGGGAACTCTGGTGACCGTCAGCTCC

The protein sequence encoded by it (115 aa):

(SEQ ID NO: 18)
EVQLVESGGGLVQPGGSLRLSCAASGFTFSDNWMNWVRQAPG
KGLEWVAQIRNKPYNYETEYAASVKGRFTISRDDSKNSAYLQMNSL
KTEDTAVYYCTAQFAYWGQGTLVTVSS

The DNA sequence of the light chain variable region (321 bp):

(SEQ ID NO: 19)
GACATTCAGATGACTCAGAGCCCTTCTTCTCTGTCCGCATCT
GTGGGAGACCGGGTCACCATCACATGCAGAGCCAGCGAGAACA
TCTACGGCGGCCTGAACTGGTATCAGCAGAAGCCAGGCAAAGC
TCCCAAGCTGCTGATCTACGGAGCAACCTCCCTGGCATCTGGAG
TGCCATCCCGGTTCAGTGGATCAGGCAGCGGGACCGACTATACT
CTGACCATTAGCTCCCTGCAGCCTGAAGACTTCGCCACATACTA
TTGCCAGAACGTGCTGAGGTCCCCATTCACCTTTGGATCTGGCA
CCAAGCTGGAGATCAAG

The protein sequence encoded by it (107 aa):

(SEQ ID NO: 20)
DIQMTQSPSSLSASVGDRVTITCRASENIYGGLNWYQQKPGKA
PKLLIYGATSLASGVPSRFSGSGSGTDYTLTISSLQPEDFATYYCQNV
LRSPFTFGSGTKLEIK

### 4. The Light Chain and Heavy Chain Sequences of the 8D2 Humanized Monoclonal Antibody 8D2H2L15

The DNA sequence of the heavy chain variable region (345 bp):

(SEQ ID NO: 13)
GAAGTGCAGCTGGTCGAGTCCGGGGGGGGCCTGGTGC
AGCCAGGAGGATCAATGCGACTGAGCTGCGCCGCTTCCGG
CTTCACCTTCAGCGACAACTGGATGAATTGGGTCAGGCAGG
CACCAGGAAAGGGACTGGAGTGGCTGGCACAGATCCGCAA
CAAACCTTACAACTACGAAACTTACTACAGCGCCTCCGTGA
AGGGGCGGTTCACCATTTCTAGAGACGATTCTAAAAACAGT
GTGTACCTGCAGATGAATAGCCTGAAGACCGAGGATACAGG
AGTCTACTATTGTACCGCACAGTTTGCTTATTGGGGGCAGG
GCACTCTGGTGACAGTCTCTTCA

The protein sequence encoded by it (115 aa):

(SEQ ID NO: 14)
EVQLVESGGGLVQPGGSMRLSCAASGFTFSDNWMNWVR
QAPGKGLEWLAQIRNKPYNYETYYSASVKGRFTISRDDSKNSV
YLQMNSLKTEDTGVYYCTAQFAYWGQGTLVTVSS

The DNA sequence of the light chain variable region (321 bp):

(SEQ ID NO: 21)
GACATCCAGATGACTCAGTCTCCCAGCTCCCTGTCCGC
TTCTGTGGGCGATCGGGTCACTATCACCTGTAGAACCAGCG
AGAACATTTACGGCGGACTGAATTGGTATCAGAGGAAGCCC
GGGAAAAGTCCTAAGCTGCTGATCTACGGAGCAACAAACCT
GGCCTCCGGCGTGTCTAGTCGCTTCAGTGGATCAGGCAGCG
GGACCGACTATACACTGACTATTTCAAGCCTGCAGCCAGAG
GATGTGGCCACATACTATTGCCAGAATGTCCTGAGCCGGCA
CCCCGGATTTGGCTCAGGGACCAAACTGGAAATTAAG

The protein sequence encoded by it (107 aa):

(SEQ ID NO: 22)
DIQMTQSPSSLSASVGDRVTITCRTSENIYGGLNWYQRKP
GKSPKLLIYGATNLASGVSSRFSGSGSGTDYTLTISSLQPEDVAT
YYCQNVLSRHPGFGSGTKLEIK

### 5. The Light Chain and Heavy Chain Sequences of the 8D2 Humanized Monoclonal Antibody 8D2H2L17

The DNA sequence of the heavy chain variable region (345 bp):

(SEQ ID NO: 13)
GAAGTGCAGCTGGTCGAGTCCGGGGGGGGCCTGGTGC
AGCCAGGAGGATCAATGCGACTGAGCTGCGCCGCTTCCGG
CTTCACCTTCAGCGACAACTGGATGAATTGGGTCAGGCAGG
CACCAGGAAAGGGACTGGAGTGGCTGGCACAGATCCGCAA
CAAACCTTACAACTACGAAACTTACTACAGCGCCTCCGTGA
AGGGGCGGTTCACCATTTCTAGAGACGATTCTAAAAACAGT
GTGTACCTGCAGATGAATAGCCTGAAGACCGAGGATACAGG
AGTCTACTATTGTACCGCACAGTTTGCTTATTGGGGGCAGG
GCACTCTGGTGACAGTCTCTTCA

The protein sequence encoded by it (115 aa):

(SEQ ID NO: 14)
EVQLVESGGGLVQPGGSMRLSCAASGFTFSDNWMNWVR
QAPGKGLEWLAQIRNKPYNYETYYSASVKGRFTISRDDSKNSV
YLQMNSLKTEDTGVYYCTAQFAYWGQGTLVTVSS

The DNA sequence of the light chain variable region (321 bp):

(SEQ ID NO: 23)
GACATCCAGATGACTCAGTCACCCAGCTCCCTGAGTG
CTTCAGTGGGCGATCGGGTCACTATCACCTGTAGAACCAGC
GAGAACATTTACGGCGGACTGAATTGGTATCAGAGGAAGCC
CGGGAAAAGCCCTAAGCTGCTGATCTACGGAGCAACAAACC
TGGCCTCCGGCGTGTCTAGTCGCTTCAGCGGCAGCGGCTCT
GGAACCGACTATACACTGACTATTTCAAGCCTGCAGCCAGA
GGATGTGGCCACATACTATTGCCAGAATGTCCTGTCCTCTC
GACCCGGATTTGGCAGTGGGACCAAACTGGAAATTAAG

The protein sequence encoded by it (107 aa):

(SEQ ID NO: 24)
DIQMTQSPSSLSASVGDRVTITCRTSENIYGGLNWYQRKP
GKSPKLLIYGATNLASGVSSRFSGSGSGTDYTLTISSLQPEDVAT
YYCQNVLSSRPGFGSGTKLEIK

### Example 4. Preparation of the 8D2 Recombinant Antibody, 8D2(Re), and the 8D2 Humanized Antibodies, 8D2H1L1, 8D2H2L3, 8D2H3L3, 8D2H2L15 and 8D2H2L17, and Detection by SDS-PAGE

### 1. Preparation of the 8D2 Recombinant Antibody, 8D2(Re), and Detection by SDS-PAGE

The cDNA sequence of the heavy chain (its variable region sequence is shown in SEQ ID NO: 5) and the cDNA sequence of the light chain (its variable region sequence is shown in SEQ ID NO: 7) of 8D2 were cloned into the pUC57simple vector (provided by Genscript Co.), respectively, resulting in the plasmids pUC57simple-8D2H and pUC57simple-8D2L.

The plasmids pUC57simple-8D2H and pUC57simple-8D2L were digested with the endonucleases (Hind III and EcoR I), respectively. The fragments encoding the heavy chain and light chain recovered via electrophoresis were separately subcloned into the pcDNA3.1 vector. The recombinant plasmids were extracted and co-transfected into cells of 293F. After 7 days of cell culture, the culture liquid was subjected to high-speed centrifugation, vacuum filtration through a microporous filter membrane and purification on a HiTrap Protein A HP column. Purified samples were added into the reductive loading buffer for protein electrophoresis and the non-reductive loading buffer for protein electrophoresis. After boiling, detection was performed by SDS-PAGE. As shown in FIG. 3, the protein of interest is shown as two band at about 50 kD and 25 kD for the reductive protein sample, or as a band at about 150 kD for the non-reductive protein sample.

### 2. Preparation of the 8D2 Humanized Antibodies, 8D2H1L1, 8D2H2L2 and 8D2H3L3, and Detection by SDS-PAGE

The cDNA sequences of the heavy chain (their variable region sequences are shown in SEQ ID NO: 9, SEQ ID NO: 13, SEQ ID NO: 17, SEQ ID NO: 13, SEQ ID NO: 13, respectively) and the cDNA sequences of the light chain (their variable region sequences are shown in SEQ ID NO: 11, SEQ ID NO: 15, SEQ ID NO: 19, SEQ ID NO: 21, SEQ ID NO: 23, respectively) of 8D2H1L1, 8D2H2L2, 8D2H3L3, 8D2H2L15, and 8D2H2L17 were cloned into the pUC57simple vector (provided by Genscript Co.), respectively, resulting in the plasmids pUC57simple-8D2H1L1, pUC57simple-8D2H2L2, pUC57simple-8D2H3L3, pUC57simple-8D2H2L15, and pUC57simple-8D2H2L17. They were separately subcloned into the pcDNA3.1 vector following the procedure described above for 8D2(Re).

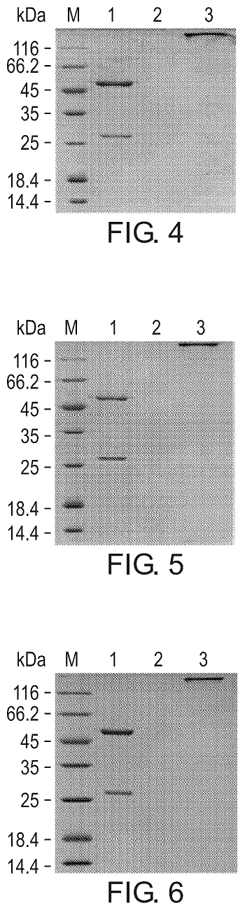

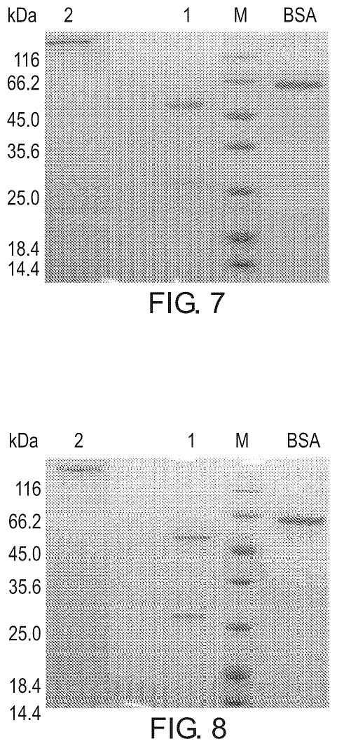

The recombinant plasmids were transfected into cells of 293F. The culture liquid of the 293F cells were subjected to detection after purification following the procedure described above for 8D2(Re). The results are shown in FIG. 4, FIG. 5, FIG. 6, FIG. 7, and FIG. 8. The reductive protein samples showed the proteins of interest as two bands at about 50 kD and 25 kD, and the non-reductive protein samples showed the proteins of interest as a band at about 150 kD.

The 8D2 recombinant antibody, 8D2(Re), and the 8D2 humanized antibodies, 8D2H1L1, 8D2H2L3, 8D2H3L3, 8D2H2L15 and 8D2H2L17 used in the following examples were prepared following the procedure described in this example.

### Example 5. Determination of the Dynamic Parameters of the Antibodies

The dynamic parameters of the binding of the antibodies 8D2 and humanized 8D2H1L1, 8D2H2L2 and 8D2H3L3 to the antigen CTLA4 (NCBI Gene ID: 1493, with the coding nucleic acid sequence as shown in SEQ ID NO: 25 and the encoded amino acid sequence as shown in SEQ ID NO: 26) were determined using the Fortebio molecular interaction analyzer.

1. The CTLA4-mFc protein (CTLA4-mFc was generated following the same method as that described in Example 1 for the synthesis of CTLA4ECD-mFc) was cleaved with the TEV protease, and the CTLA4 antigen was obtained by purification on a column.

The sequence of the CTLA4 gene (636 bp):

(SEQ ID NO: 25)
ATGGGCGTCCTGCTGACTCAGAGAACCCTGCTGTCCCTGGT
GCTGGCACTGCTGTTTCCTTCAATGGCTTCAATGGCTATGCATG
TGGCTCAGCCAGCAGTGGTCCTGGCAAGCTCCAGGGGGATCGC
CAGTTTCGTGTGCGAGTACGCCTCACCTGGAAAGGCTACAGAAG
TCCGGGTGACTGTCCTGAGACAGGCTGACTCTCAGGTGACCGA
GGTCTGCGCCGCTACATATATGATGGGCAACGAACTGACCTTTC
TGGACGATTCCATTTGTACTGGCACCTCTAGTGGGAACCAAGTG
AATCTGACTATCCAGGGACTGCGAGCAATGGACACCGGACTGTA
CATTTGCAAAGTGGAGCTGATGTATCCCCCTCCATACTATCTGG
GCATCGGGAATGGAACACAGATCTACGTGATTGATCCCGAACCT
TGTCCAGACAGCGATTTCCTGCTGTGGATTCTGGCAGCCGTGTC
AAGCGGCCTGTTCTTTTATAGCTTTCTGCTGACTGCCGTCTCCCT
GTCTAAGATGCTGAAGAAACGATCCCCCCTGACCACAGGGGTG
GTCGTGAAAATGCCACCTACCGAGCCCGAGTGCGAAAAACAGTT
CCAGCCATACTTTATCCCTATCAAT

The encoded corresponding amino acid sequence (212 aa):

(SEQ ID NO: 26)
MGVLLTQRTLLSLVLALLFPSMASMAMHVAQPAVVLASSRGI
ASFVCEYASPGKATEVRVTVLRQADSQVTEVCAATYMMGNELTFL
DDSICTGTSSGNQVNLTIQGLRAMDTGLYICKVELMYPPPYYLGIG
NGTQIYVIDPEPCPDSDFLLWILAAVSSGLFFYSFLLTAVSLSKMLK
KRSPLTTGVVVKMPPTEPECEKQFQPYFIPIN

2. The antibody 8D2 and its humanized antibodies 8D2H1L1, 8D2H2L2, 8D2H3L3, 8D2H2L15, and 8D2H2L17 were immobilized on the surface of the AR2G sensor by amino coupling, and blocked with ethanolamine. After equilibration in PBST, the CTLA4 antigen was added for binding. CTLA4 was serially 2× diluted in PBST, and the following concentrations were obtained: 300, 150, 75, 37.5, 18.75, 9.38, 4.69, 0 nM. Dissociation occurred in PBST. The humanized antibodies 8D2H1L1, H2L2, H3L3, H2L15, and H2L17 were detected by a method same as 8D2, and the antigen concentrations were 180, 90, 45, 22.5, 11.25, 5.625, 2.813, 0 nM.

The dynamic parameters of the antibody 8D2 and its humanized antibodies 8D2H1L1, 8D2H2L2, 8D2H3L3, 8D2H2L15, and 8D2H2L17 are provided in Table 1.

TABLE 1
Dynamic parameters of the antibodies 8D2, 8D2H1L1,
8D2H2L2, 8D2H3L3, 8D2H2L15, and 8D2H2L17

| Antibody | K_{D} | kₒₙ | kₒₙ | k_{dis} | k_{dis} |
|---|---|---|---|---|---|
| Name | (M) | (1/Ms) | Error | (1/s) | Error |
| 8D2 | 1.66E−10 | 1.42E+05 | 1.22E+03 | 2.36E−05 | 2.09E−06 |
| 8D2H1L1 | 6.08E−10 | 3.40E+05 | 1.17E+04 | 2.07E−04 | 1.81E−05 |
| 8D2H2L2 | 9.55E−10 | 4.07E+05 | 1.59E+04 | 3.88E−04 | 1.60E−05 |
| 8D2H3L3 | 1.05E−09 | 3.12E+05 | 1.01E+04 | 3.27E−04 | 1.41E−05 |
| 8D2H2L15 | 1.02E−09 | 4.54E+05 | 8.18E+03 | 4.65E−04 | 9.50E−06 |
| 8D2H2L17 | 7.66E−10 | 4.59E+05 | 8.21E+03 | 3.52E−04 | 8.30E-06 |
| 10D1 | 1.21E−09 | 4.67E+05 | 1.15E+04 | 5.65E−04 | 1.51E−05 |
| 11.2.1 | 9.03E−10 | 3.87E+05 | 5.46E+03 | 3.49E−04 | 7.32E−06 |

K_{D}, affinity constant;
kₒₙ, antigen-antibody association rate;
k_{dis}, antigen- antibody dissociation rate;
K_{D} = k_{dis}/kₒₙ.

The results demonstrate that all of the six antibodies have a good affinity for the antigen, which is comparable or even superior than the control antibodies 10D1 and 11.2.1.

### Example 6. Determination of the Activity of the Antibodies to Bind to the Antigen CTLA4 on the Surface of the Hybridoma Cell Line by Flow Cytometry

First, 293F host cells expressing the CTLA4 antigen were generated, and labeled with the monoclonal antibody 8D2 (Example 1) and 8D2(Re) and the 8D2 humanized antibodies 8D2H1L1, 8D2H2L2 and 8D2H3L3 (Example 4) prepared in the present invention, respectively. Then, the ability of the antibodies to specifically bind to the antigen having native conformation on the surface of cells was verified by flow cytometry.

The specific steps are provided below.

### 1. Generation of 293F Host Cells Expressing the CTLA4 Antigen

Cells of 293F were transfected with the plasmid pLenti6.3-CTLA4 for CTLA4 (the vector pLenti6.3 was purchased from Invitrogen Co.) using the Lipofectamin transfection kit (purchased from Invitrogen Co.). After screening, a clonal population of cells stably expressing CTLA4 (293F-CTLA4) was obtained.

2. Labeling with the Antibodies and Detection Using a Flow Cytometer

The 293F host cells expressing the CTLA4 antigen obtained by the above steps were digested with trypsin following a conventional method, and 2×10⁵ cells were added to each collection tube. The diluted solutions of the 8D2 antibody in PBS containing 1% BSA were prepared to achieve the concentrations of 20 nM, 10 nM, 5 nM, 1 nM, 0.1 nM, 0.01 nM, and 0 nM, respectively. After incubation with 293F cells expressing CTLA4 on ice for 2 hours, 100 μL FITC-Goat-Anti-Mouse IgG (1:500) was added to each tube, and the tubes were incubated on ice for 1 hour. After addition of 300 μL PBS, the fluorescent signal was detected using the FITC channel on the flow cytometer. Other antibodies were detected in a similar way to the 8D2 antibody.

### 3. Results

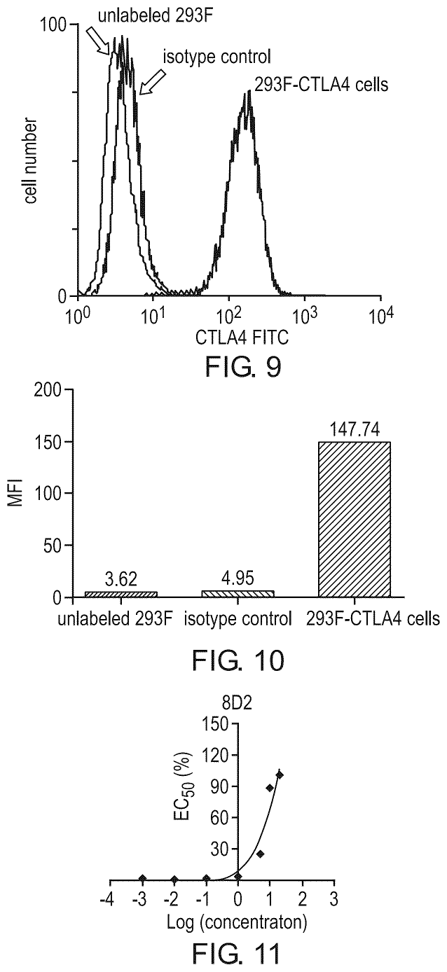

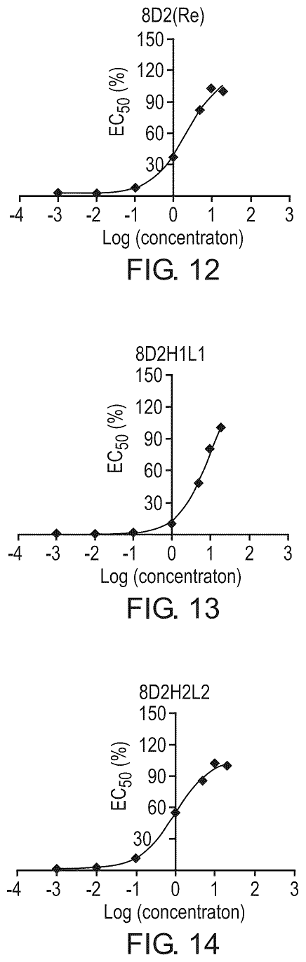

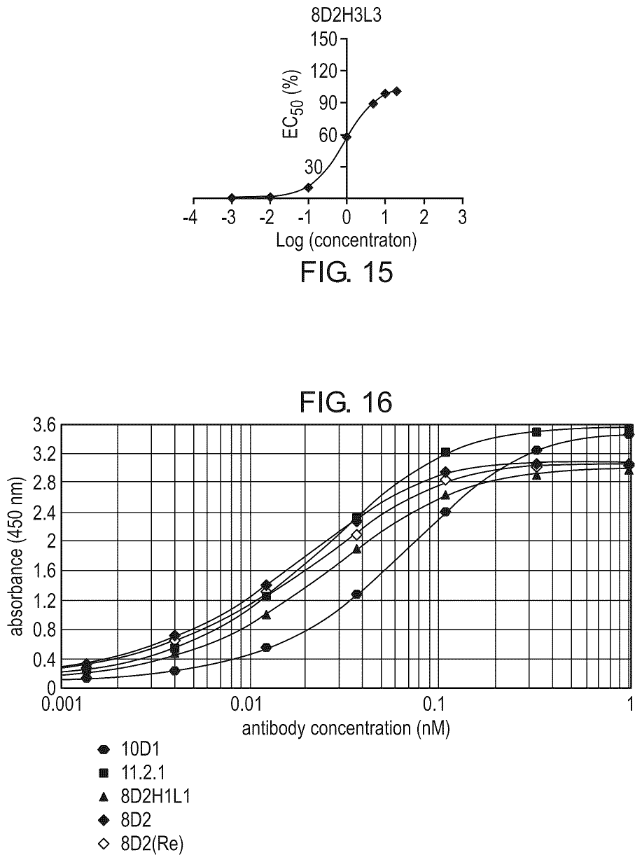

The results of verifying the expression of CTLA4 on 293F-CTLA4 cells are shown in FIG. 9 and FIG. 10, respectively. The results of the binding of the antibodies 8D2, 8D2(Re), and the three humanized antibodies to 293F cells are shown in FIGS. 11 to 15, respectively. As shown in the figures, the 8D2 antibody and its humanized antibodies can effectively bind to the CTLA4 target protein on the surface of the 293F host cell, and their binding efficiency is dose dependent. The fluorescent intensities at each dose are provided in Table 2.

The binding efficiency, EC₅₀, of 8D2 and its humanized antibodies was obtained by curve simulation in the fluorescent quantitative analysis of the bound antibodies 8D2 and its humanized antibodies, which is shown in Table 3.

TABLE 2
Fluorescent intensity analysis determining the binding
of 8D2, 8D2(Re) and the 8D2 humanized antibodies 8D2H1L1,
8D2H2L2 and 8D2H3L3 to the CTLA4 antigen on the surface
of the 293F-CTLA4 host cell by flow cytometry

| concen- | | | | | |
|---|---|---|---|---|---|
| tration | 8D2 | 8D2(Re) | 8D2H1L1 | 8D2H2L2 | 8D2H3L3 |
| (nM) | fluorescence intensity | | | | |
| 0.001 | 7.60 | 24.62 | 10.84 | 10.85 | 10.85 |
| 0.01 | 7.70 | 24.72 | 10.85 | 32.48 | 25.14 |
| 0.1 | 9.10 | 66.72 | 21.25 | 124.03 | 108.29 |
| 1 | 25.50 | 321.27 | 103.04 | 624.65 | 623.25 |
| 5 | 182.60 | 713.87 | 558.75 | 972.03 | 970.80 |
| 10 | 638.60 | 897.63 | 943.84 | 1159.24 | 1084.74 |
| 25 | 721.80 | 873.24 | 1170.64 | 1132.39 | 1091.77 |

TABLE 3
The binding efficiency, EC₅₀, of 8D2, 8D2(Re) and the 8D2
humanized antibodies 8D2H1L1, 8D2H2L2 and 8D2H3L3 to the
CTLA4 antigen on the surface of the 293F-CTLA4 host cell
obtained by curve simulation in the analysis by flow cytometry

| | 8D2 | 8D2(Re) | 8D2H1L1 | 8D2H2L2 | 8D2H3L3 |
|---|---|---|---|---|---|
| EC₅₀ | 3.84 | 1.38 | 5.06 | 4.37 | 4.54 |
| (nM) | | | | | |

The results demonstrate that the antibodies 8D2, 8D2(Re) and the 8D2 humanized antibodies 8D2H1L1, 8D2H2L2 and 8D2H3L3 all have a very strong capability to bind to the CTLA4 antigen on the surface of the 293F-CTLA4 host cells.

### Example 7. Determination of the Activity of the Antibodies to Bind to the CTLA4 Antigen by ELISA

The ELISA plate was coated with CTLA4 at 4° C. over night. After blocking with 1% BSA at 37° C. for 2 h, the CTLA4 antibodies 8D2, 8D2(Re) and the 8D2 humanized antibodies 8D2H1L1, 8D2H2L2, 8D2H3L3, 8D2H2L15, and 8D2H2L17, and the control antibodies 10D1 (Alan J. Korman, Edward L. Halk, et al., HUMAN CTLA-4 ANTIBODIES, U.S. Pat. No. 6,984,720 B1) and 11.2.1 (Douglas Charles Hanson, Mark Joseph Neveu, et al., Human monoclonal antibodies to CTLA-4, U.S. Pat. No. 682,736 B1) were added for reaction for 30 min. The enzyme conjugated secondary antibody was added for incubation for 30 min. Then, the absorbance at 450 nm was determined on an ELISA plate reader.

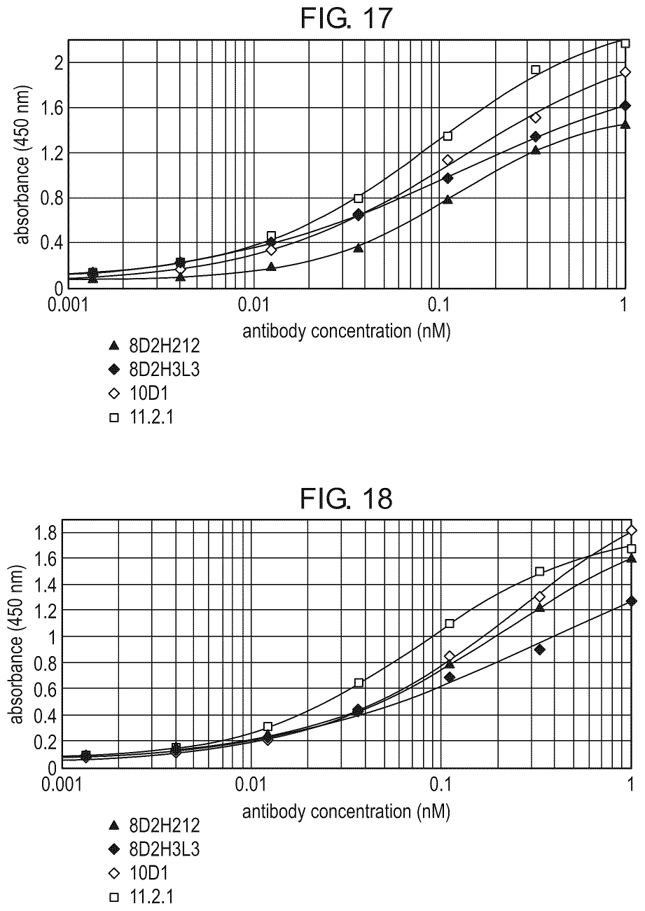

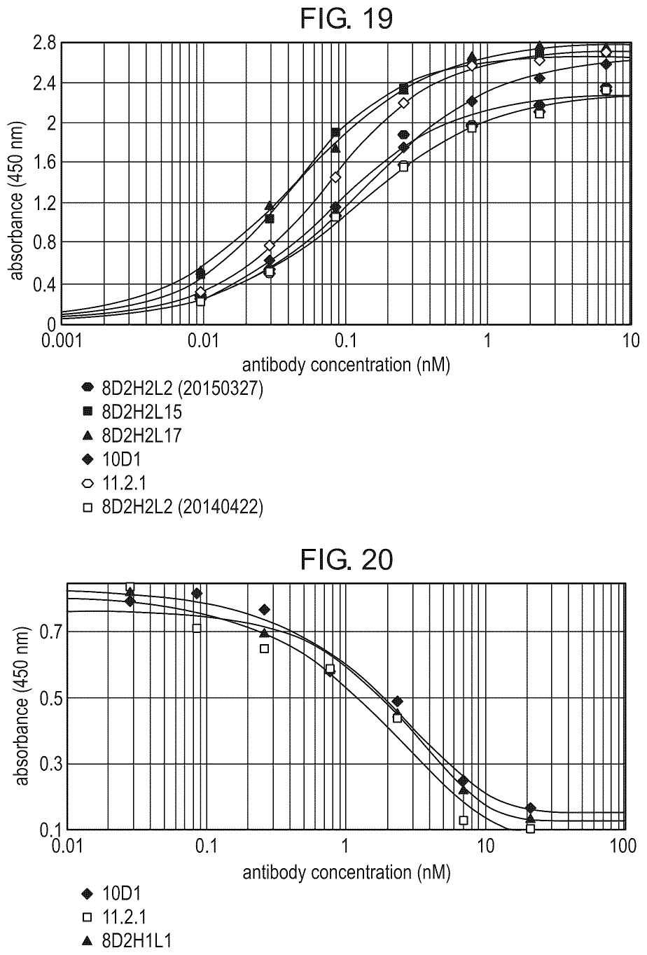

The results of detecting the binding of the 8D2 antibody and its humanized antibodies to the CTLA4 antigen are shown in FIGS. 14 to 19, respectively. As shown in the figures, the antibodies 8D2, 8D2(Re) and the 8D2 humanized antibodies all can effectively bind to the CTLA4 protein, and their binding efficiency is dose dependent. The fluorescent intensities at each dose are provided in Tables 4 to 8. The binding efficiency, EC₅₀, of 8D2, 8D2(Re) and the humanized antibodies was obtained by curve simulation in the fluorescent quantitative analysis of the bound 8D2, 8D2(Re) and the humanized antibodies (Table 9).

TABLE 4
Binding of 8D2 and 8D2(Re) to murine CTLA4 (ELISA)

| Antibody | | | | | | |
|---|---|---|---|---|---|---|
| concen- | | | | | | |
| tration | Antigen coating: murine CTLA4 at 0.5 μg/ml | | | | | |
| (μg/ml) | 8D2 | | 8D2(Re) | | 10D1 | |
| 1 | 2.823 | 2.682 | 2.672 | 2.769 | 2.995 | 2.975 |
| 0.3 | 2.806 | 2.763 | 2.690 | 2.735 | 2.852 | 2.900 |
| 0.1 | 2.754 | 2.718 | 2.796 | 2.685 | 2.429 | 2.538 |
| 0.03 | 2.336 | 2.381 | 2.305 | 2.259 | 1.507 | 1.704 |
| 0.01 | 1.614 | 1.560 | 1.397 | 1.446 | 0.673 | 0.794 |
| 0.003 | 0.784 | 0.760 | 0.662 | 0.674 | 0.292 | 0.328 |
| 0.001 | 0.358 | 0.355 | 0.315 | 0.321 | 0.136 | 0.142 |
| 0 | 0.063 | 0.052 | 0.053 | 0.046 | 0.046 | 0.050 |

| | | |
|---|---|---|
| Secondary | Goat Anti Mouse | Goat Anti Human |
| antibody | Secondary Antibody | Secondary Antibody |

TABLE 5
Binding of 8D2, 8D2H1L1 and 8D2(Re) to human CTLA4 (ELISA)

| Antibody | | | | | | | | | | |
|---|---|---|---|---|---|---|---|---|---|---|
| concen- | | | | | | | | | | |
| tration | Antigen coating: human CTLA4 at 0.5 μg/ml | | | | | | | | | |
| (μg/ml) | 10D1 | | 11.2.1 | | 8D2H1L1 | | 8D2 | | 8D2(Re) | |
| 1 | 3.479 | 3.432 | 3.584 | 3.547 | 3.016 | 3.031 | 3.029 | 3.107 | 3.058 | 3.085 |
| 1:3 | 3.323 | 3.155 | 3.499 | 3.479 | 2.834 | 2.904 | 3.076 | 3.074 | 2.930 | 3.072 |
| 1:9 | 2.506 | 2.293 | 3.211 | 3.187 | 2.610 | 2.670 | 2.878 | 2.988 | 2.805 | 2.868 |
| 1:27 | 1.331 | 1.194 | 2.337 | 2.293 | 1.834 | 1.944 | 2.265 | 2.287 | 2.052 | 2.064 |
| 1:81 | 0.552 | 0.528 | 1.254 | 1.267 | 0.969 | 0.996 | 1.335 | 1.479 | 1.398 | 1.271 |
| 1:243 | 0.202 | 0.222 | 0.536 | 0.552 | 0.450 | 0.515 | 0.666 | 0.770 | 0.634 | 0.649 |
| 1:729 | 0.141 | 0.115 | 0.253 | 0.263 | 0.204 | 0.206 | 0.277 | 0.351 | 0.307 | 0.309 |
| 0 | 0.090 | 0.086 | 0.072 | 0.064 | 0.067 | 0.067 | 0.064 | 0.067 | 0.071 | 0.086 |

| | | |
|---|---|---|
| Secondary | Goat Anti Human IgG | Goat Anti Mouse IgG |
| Antibody | Secondary antibody | Secondary Antibody |

TABLE 6
Binding of 8D2H2L2 and 8D2H3L3 to human CTLA4 (ELISA)

| Antibody | | | | | | | | |
|---|---|---|---|---|---|---|---|---|
| concen- | | | | | | | | |
| tration | Antigen coating: human CTLA4 at 0.5 μg/ml | | | | | | | |
| (μg/ml) | 8D2H2L2 | | 8D2H3L3 | | 10D1 | | 11.2.1 | |
| 1 | 1.489 | 1.411 | 1.631 | 1.601 | 1.775 | 2.069 | 2.206 | 2.150 |
| 1:3 | 1.178 | 1.262 | 1.192 | 1.455 | 1.527 | 1.480 | 1.825 | 2.047 |
| 1:9 | 0.710 | 0.872 | 0.943 | 1.007 | 1.073 | 1.204 | 1.292 | 1.409 |
| 1:27 | 0.336 | 0.370 | 0.642 | 0.658 | 0.663 | 0.585 | 0.893 | 0.682 |
| 1:81 | 0.192 | 0.195 | 0.415 | 0.374 | 0.349 | 0.323 | 0.499 | 0.426 |
| 1:243 | 0.097 | 0.109 | 0.230 | 0.214 | 0.132 | 0.146 | 0.223 | 0.219 |
| 1:729 | 0.075 | 0.083 | 0.100 | 0.130 | 0.099 | 0.099 | 0.127 | 0.136 |
| 0 | 0.052 | 0.055 | 0.052 | 0.057 | 0.056 | 0.053 | 0.057 | 0.061 |

| | |
|---|---|
| Secondary | HRP Conjugated Goat Anti Human IgG |
| antibody | Secondary Antibody |

TABLE 7
Binding of 8D2H2L2 and 8D2H3L3 to monkey CTLA4 (ELISA)

| Antibody | | | | | | | | |
|---|---|---|---|---|---|---|---|---|
| concen- | | | | | | | | |
| tration | Antigen coating: monkey CTLA4-hFc at 0.25 μg/ml | | | | | | | |
| (μg/ml) | 8D2H2L2 | | 8D2H3L3 | | 10D1 | | 11.2.1 | |
| 1 | 1.576 | 1.624 | 1.235 | 1.321 | 1.788 | 1.846 | 1.718 | 1.632 |
| 1:3 | 1.223 | 1.199 | 0.921 | 0.873 | 1.250 | 1.344 | 1.540 | 1.460 |
| 1:9 | 0.793 | 0.775 | 0.654 | 0.724 | 0.845 | 0.868 | 1.114 | 1.054 |
| 1:27 | 0.471 | 0.426 | 0.441 | 0.403 | 0.429 | 0.402 | 0.625 | 0.665 |
| 1:81 | 0.220 | 0.230 | 0.239 | 0.218 | 0.190 | 0.191 | 0.297 | 0.313 |
| 1:243 | 0.114 | 0.117 | 0.123 | 0.119 | 0.104 | 0.108 | 0.130 | 0.172 |
| 1:729 | 0.071 | 0.076 | 0.088 | 0.096 | 0.063 | 0.067 | 0.082 | 0.094 |
| 0 | 0.048 | 0.048 | 0.048 | 0.050 | 0.049 | 0.053 | 0.048 | 0.051 |

| | |
|---|---|
| Secondary | HRP Conjugated Goat Anti Human IgG, F(ab′)₂ |
| antibody | Secondary Antibody |

TABLE 8
Binding of 8D2H2L15 and 8D2H2L17 to human CTLA4 (ELISA)

| Antibody | | | | | | | | | | | | |
|---|---|---|---|---|---|---|---|---|---|---|---|---|
| concen- | Antigen coating: CTLA4 at 0.5 μg/ml | | | | | | | | | | | |
| tration | 8D2H2L2 | | | | | | | | | | 8D2H2L2 | |
| (μg/ml) | 20150327 | | 8D2H2L15 | | 8D2H2L17 | | 10D1 | | 11.2.1 | | 20140422 | |
| 1 | 2.34 | 2.37 | 2.58 | 2.55 | 2.61 | 2.81 | 2.56 | 2.74 | 2.75 | 2.69 | 2.23 | 2.40 |
| 1:3 | 2.22 | 2.09 | 2.65 | 2.72 | 2.73 | 2.78 | 2.42 | 2.44 | 2.56 | 2.66 | 2.09 | 2.07 |
| 1:9 | 2.03 | 1.87 | 2.79 | 2.45 | 2.59 | 2.73 | 2.20 | 2.20 | 2.69 | 2.44 | 1.92 | 1.95 |
| 1:27 | 1.82 | 1.93 | 2.43 | 2.21 | 2.41 | 2.28 | 1.81 | 1.70 | 2.13 | 2.28 | 1.47 | 1.63 |
| 1:81 | 1.10 | 1.17 | 1.95 | 1.83 | 1.80 | 1.68 | 1.03 | 1.09 | 1.37 | 1.53 | 1.10 | 1.01 |
| 1:243 | 0.65 | 0.58 | 1.05 | 1.02 | 1.14 | 1.19 | 0.51 | 0.53 | 0.75 | 0.79 | 0.49 | 0.50 |
| 1:729 | 0.26 | 0.21 | 0.53 | 0.44 | 0.57 | 0.50 | 0.21 | 0.24 | 0.32 | 0.31 | 0.23 | 0.20 |
| 0 | 0.04 | 0.05 | 0.05 | 0.04 | 0.04 | 0.05 | 0.04 | 0.05 | 0.05 | 0.05 | 0.05 | 0.05 |

Secondary Antibody: HRP Conjugated Goat Anti Human IgG (1:5000)

TABLE 9
The binding efficiency, EC₅₀, of 8D2, 8D2(Re) and the
8D2 humanized antibodies 8D2H1L1, 8D2H2L2, 8D2H3L3,
8D2H2L15, and 8D2H2L17 to the CTLA4 antigen obtained
by curve simulation in the analysis by ELISA

| | | Antibody | 10D1 | 11.2.1 |
|---|---|---|---|---|
| | Source of the | EC₅₀ | EC₅₀ | EC₅₀ |
| | CTLA4 antigen | (nM) | (nM) | (nM) |
| 8D2 | Mouse | 0.015 | 0.062 | 0.023 |
| | | 0.071 | 0.24 | |
| 8D2(Re) | Mouse | 0.015 | 0.062 | 0.023 |
| | | 0.085 | 0.24 | |
| 8D2H1L1 | Mouse | 0.025 | 0.062 | 0.023 |
| 8D2H2L2 | Human | 0.12 | 0.125 | 0.09 |
| 8D2H2L2 | Human | 0.082 | 0.125 | 0.09 |
| 8D2H2L2 | Human | 0.118 | 0.125 | 0.09 |
| 8D2H3L3 | Human | 0.129 | 0.125 | 0.09 |
| 8D2H2L2 | Monkey | 0.227 | 0.258 | 0.075 |
| 8D2H3L3 | Monkey | 0.385 | 0.258 | 0.075 |
| 8D2H2L15 | Human | 0.042 | 0.138 | 0.075 |
| 8D2H2L17 | Human | 0.047 | 0.138 | 0.075 |

Note:
8D2H2L2 was measured in triplicate.

The above results demonstrate that the antibodies 8D2 and 8D2(Re) bind to the murine CTLA4 antigen with an efficiency better than that of the control antibodies 10D1 and 11.2.1. The humanized antibody 8D2H1L1 binds to the murine CTLA4 antigen with an efficiency stronger than that of the control antibody 10D1 and comparable to that of 11.2.1.

The humanized antibody 8D2H2L2 binds to the human CTLA4 antigen with an efficiency comparable to that of 10D1. The humanized antibodies 8D2H2L2 and 8D2H3L3 bind to the monkey CTLA4 antigen with an efficiency comparable to that of 10D1. The humanized antibody 8D2H2L15 and 8D2H2L17 bind to the human CTLA4 antigen with an efficiency significantly stronger than that of the control antibodies 10D1 and 11.2.1.

### Example 8. Detection of the Activity of the Antibodies to Compete with B7-1/2 for Binding to the CTLA4 Antigen by Competitive ELISA

1. Detection of the Activity of the Antibodies to Compete with B7-1 for Binding to the CTLA4 Antigen by ELISA

The ELISA plates were coated with B7-1 at 4° C. overnight. After blocking with 1% BSA at 37° C. for 2 h, the anti-CTLA4 antibodies, i.e., the monoclonal antibodies 8D2 and 8D2(Re) and the 8D2 humanized antibodies 8D2H1L1, 8D2H2L2, 8D2H3L3, 8D2H2L15, and 8D2H2L17, as well as the control antibodies 10D1 and 11.2.1 were added. After incubation for 10 minutes, CTLA4-mFc was added. After incubation at 37° C. for 40 minutes, the enzyme conjugated secondary antibody was added. After incubation at 37° C. for 30 minutes, the absorbance at 450 nm was detected on an ELISA plate reader.

2. Detection of the Activity of the Antibodies to Compete with B7-2 for Binding to the CTLA4 Antigen by ELISA

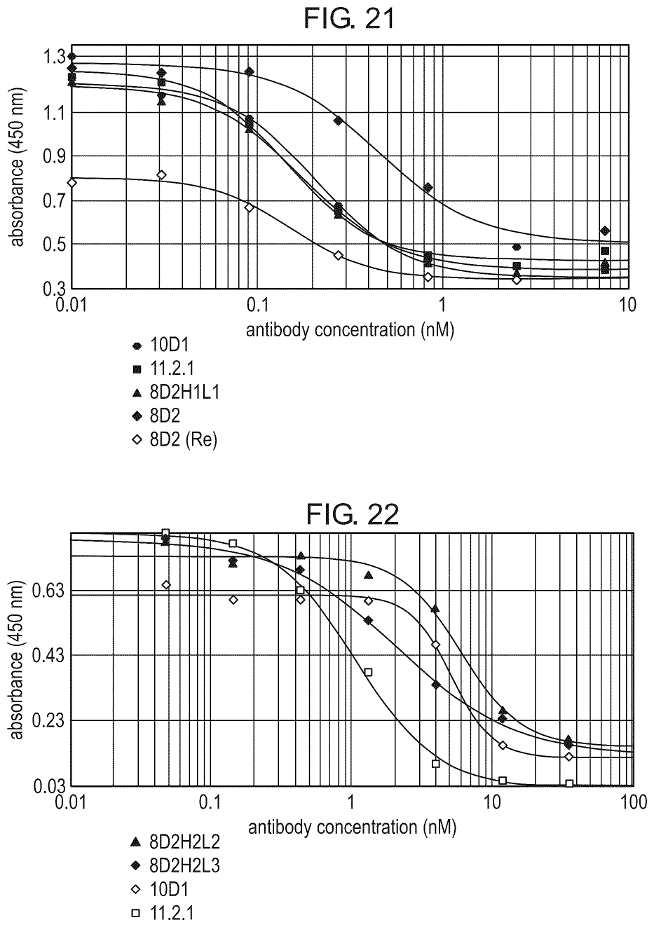

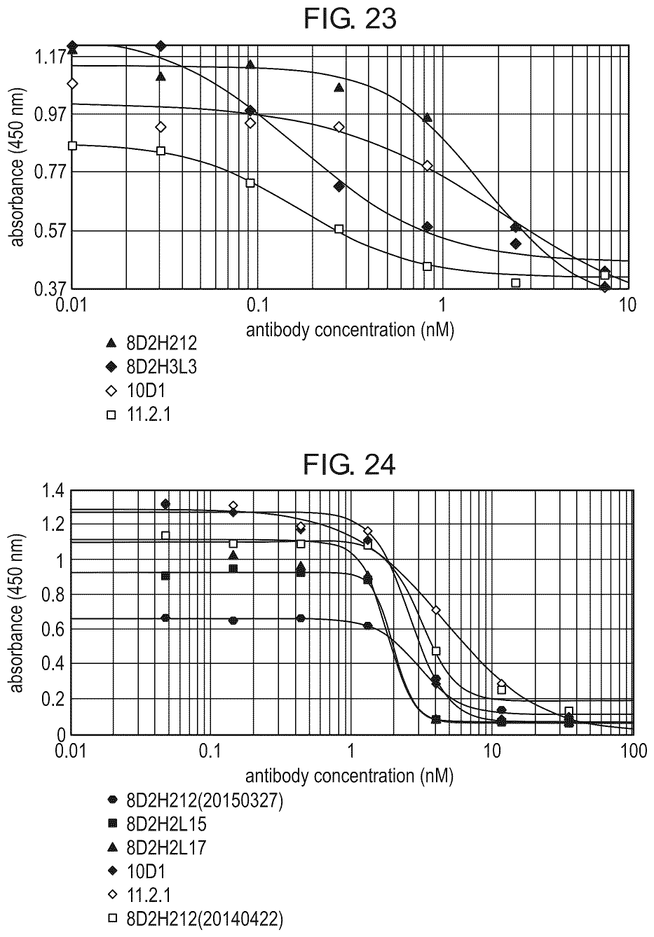

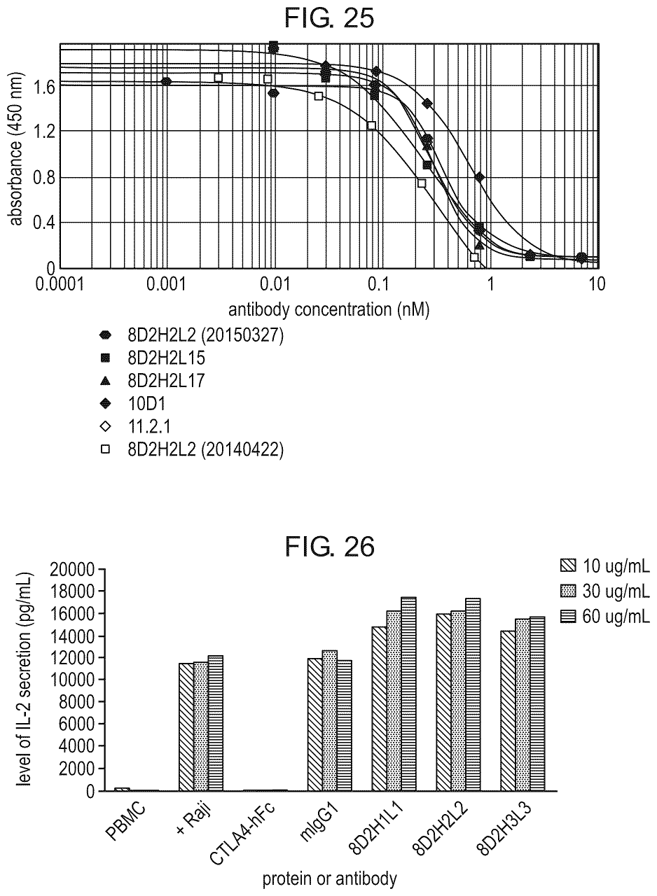

The ELISA plates were coated with CTLA4-mFc at 4° C. overnight. After blocking with 1% BSA at 37° C. for 2 h, the anti-CTLA4 antibodies, i.e., the monoclonal antibodies 8D2 and 8D2(Re) and the 8D2 humanized antibodies 8D2H1L1, 8D2H2L2, 8D2H3L3, 8D2H2L15, and 8D2H2L17, as well as the control antibodies 10D1 and 11.2.1 were added. After incubation for 10 minutes, B7-2-his was added. After incubation at 37° C. for 40 minutes, the enzyme conjugated secondary antibody was added. After incubation at 37° C. for 30 minutes, the absorbance at 450 nm was detected on an ELISA plate reader. The results of detecting the binding of the 8D2, 8D2(Re) and humanized antibodies to the CTLA4 antigen are shown in FIGS. 20 to 25, respectively. As shown in the figures, the 8D2, 8D2(Re) antibodies and the 8D2 humanized antibodies could effectively bind to the CTLA protein, and their binding efficiency is dose dependent. The fluorescent intensities at each dose are provided in Tables 10 to 16. The binding efficiency, EC₅₀, of 8D2, 8D2(Re) and the humanized antibodies was obtained by curve simulation in the fluorescent quantitative analysis of the bound antibodies 8D2, 8D2(Re) and the humanized antibodies (Table 17).

TABLE 10
8D2 and 8D2(Re) compete with B7-1 in ELISA

| Antibody concentration | Antigen coating: CTLA4-mFc at 0.2 μg/ml | | | |
|---|---|---|---|---|
| (μg/ml) | 8D2 | | 8D2(Re) | |
| 3 | 0.163 | 0.149 | 0.176 | 0.215 |
| 1 | 0.208 | 0.188 | 0.200 | 0.214 |
| 0.3 | 0.354 | 0.347 | 0.355 | 0.390 |
| 0.1 | 0.680 | 0.695 | 0.668 | 0.721 |
| 0.03 | 1.378 | 1.262 | 1.430 | 1.708 |
| 0.01 | 1.758 | 1.612 | 1.630 | 1.824 |
| 0.003 | 1.982 | 1.711 | 1.890 | 1.937 |
| 0 | 2.228 | 1.766 | 1.805 | 1.779 |

B7/1-hFc (0.3 μg/ml)

| | |
|---|---|
| Secondary antibody | Goat Anti Human Secondary Antibody |

TABLE 11
8D2, 8D2H1L1 and 8D2(Re) compete with B7-1 in ELISA

| Antibody | | | | | | | | |
|---|---|---|---|---|---|---|---|---|
| concen- | Coating: B7/1-hFc at 0.2 μg/ml | | | | | | | |
| tration | | | | | | | CTLA4-mFc | |
| (μg/ml) | 10D1 | | 11.2.1 | | 8D2 H1L1 | | (0.6 μg/ml) 1:2 | |
| 3 | 0.168 | 0.158 | 0.101 | 0.105 | 0.123 | 0.138 | 0.824 | 0.791 |
| 1:3 | 0.258 | 0.232 | 0.119 | 0.133 | 0.206 | 0.231 | 0.640 | 0.768 |
| 1:9 | 0.515 | 0.466 | 0.381 | 0.485 | 0.445 | 0.529 | 0.750 | 0.717 |
| 1:27 | 0.577 | 0.508 | 0.597 | 0.579 | 0.509 | 0.659 | 0.653 | 0.626 |
| 1:81 | 0.801 | 0.730 | 0.650 | 0.613 | 0.669 | 0.723 | 0.571 | 0.522 |
| 1:243 | 0.814 | 0.848 | 0.900 | 0.520 | 0.841 | 0.821 | 0.459 | 0.327 |
| 1:729 | 0.854 | 0.732 | 0.993 | 0.841 | 0.848 | 0.822 | 0.312 | 0.232 |
| 0 | 0.856 | 0.812 | 0.826 | 0.550 | 0.672 | 0.600 | 0.071 | 0.074 |

| | | |
|---|---|---|
| Antigen | CTLA4-mFc 0.3 μg/ml | Control |

| | |
|---|---|
| Second | HRP Conjugated Goat Anti Mouse IgG |
| antibody | Second Antibody |

TABLE 12
8D2, 8D2H1L1 and 8D2(Re) compete with B7-2 in ELISA

| Antibody | | | | | | | | | | |
|---|---|---|---|---|---|---|---|---|---|---|
| concen- | | | | | | | | | | |
| tration | Antigen coating: CTLA4-mFc at 0.5 μg/ml | | | | | | | | | |
| μg/ml | 10D1 | | 11.2.1 | | 8D2 H1L1 | | 8D2 | | 8D2(Re) | |
| 3 | 0.569 | 0.550 | 0.492 | 0.442 | 0.450 | 0.384 | 0.407 | 0.336 | 0.367 | 0.375 |
| 1:3 | 0.500 | 0.466 | 0.387 | 0.402 | 0.404 | 0.332 | 0.359 | 0.306 | 0.331 | 0.289 |
| 1:9 | 0.736 | 0.782 | 0.412 | 0.482 | 0.467 | 0.371 | 0.456 | 0.355 | 0.384 | 0.315 |
| 1:27 | 0.982 | 1.137 | 0.676 | 0.585 | 0.671 | 0.633 | 0.675 | 0.675 | 0.464 | 0.443 |
| 1:81 | 1.196 | 1.355 | 1.120 | 0.965 | 1.038 | 1.007 | 1.091 | 1.050 | 0.713 | 0.622 |
| 1:243 | 1.171 | 1.380 | 1.237 | 1.214 | 1.215 | 1.069 | 1.154 | 1.172 | 0.862 | 0.766 |
| 1:729 | 1.307 | 1.388 | 1.362 | 1.229 | 1.231 | 1.253 | 1.242 | 1.264 | 0.826 | 0.725 |
| 0 | 1.030 | 1.171 | 1.187 | 1.100 | 1.130 | 1.076 | 1.034 | 1.183 | 0.915 | 0.861 |
| Receptor | B7/2-His at 1 μg/ml | | | | | | | | | |
| Secondary | HRP Conjugated Mouse Anti His Secondary Antibody | | | | | | | | | |
| antibody | | | | | | | | | | |

TABLE 13
The 8D2H2L2 and 8D2H3L3 antibodies
compete with B7-1 in ELISA

| Antibody | | | | | | | | |
|---|---|---|---|---|---|---|---|---|
| concen- | | | | | | | | |
| tration | Coating: B7/1-hFc at 0.3 μg/ml | | | | | | | |
| (μg/ml) | 8D2 H2L2 | | 8D2 H3L3 | | 10D1 | | 11.2.1 | |
| 5 | 0.207 | 0.232 | 0.187 | 0.202 | 0.166 | 0.172 | 0.080 | 0.089 |
| 1:3 | 0.346 | 0.267 | 0.286 | 0.327 | 0.210 | 0.194 | 0.090 | 0.097 |
| 1:9 | 0.625 | 0.702 | 0.416 | 0.388 | 0.486 | 0.548 | 0.160 | 0.138 |
| 1:27 | 0.577 | 0.727 | 0.590 | 0.503 | 0.673 | 0.621 | 0.488 | 0.369 |
| 1:81 | 0.830 | 0.743 | 0.747 | 0.617 | 0.663 | 0.647 | 0.698 | 0.660 |
| 1:243 | 0.707 | 0.760 | 0.673 | 0.768 | 0.652 | 0.775 | 0.755 | 0.900 |
| 1:729 | 0.780 | 0.882 | 0.840 | 0.842 | 0.705 | 0.691 | 0.909 | 0.793 |
| 0 | 0.577 | 0.752 | 0.632 | 0.745 | 0.732 | 0.909 | 0.683 | 0.735 |

| | |
|---|---|
| Antigen | CTLA4-mFc at 0.3 μg/ml |
| Secondary | HRP Conjugated Goat Anti Mouse IgG |
| antibody | Secondary Antibody |

TABLE 14
The 8D2H2L2 and 8D2H3L3 antibodies compete
with B7-2 for binding to CTLA4 in ELISA

| Antibody | | | | | | | | |
|---|---|---|---|---|---|---|---|---|
| concen- | | | | | | | | |
| tration | Antigen Coating: CTLA4-mFc at 0.5 μg/ml | | | | | | | |
| (μg/ml) | 8D2 H2L2 | | 8D2 H3L3 | | 10D1 | | 11.2.1 | |
| 1.5 | 0.377 | 0.376 | 0.417 | 0.432 | 0.449 | 0.408 | 0.372 | 0.494 |
| 1:3 | 0.616 | 0.537 | 0.540 | 0.511 | 0.553 | 0.602 | 0.437 | 0.348 |
| 1:9 | 0.988 | 0.927 | 0.548 | 0.614 | 0.806 | 0.788 | 0.479 | 0.412 |
| 1:27 | 1.085 | 1.038 | 0.717 | 0.728 | 0.969 | 0.890 | 0.622 | 0.529 |
| 1:81 | 1.227 | 1.059 | 1.010 | 0.951 | 0.974 | 0.916 | 0.805 | 0.649 |
| 1:243 | 1.136 | 1.066 | 1.255 | 1.160 | 0.935 | 0.921 | 0.930 | 0.754 |
| 1:729 | 1.218 | 1.158 | 1.239 | 1.162 | 1.108 | 1.045 | 0.981 | 0.746 |
| 0 | 1.094 | 1.068 | 1.198 | 1.214 | 1.082 | 1.047 | 0.987 | 0.819 |

| | |
|---|---|
| Ligand | B7/2-His at 1 μg/ml |
| Secondary | HRP Conjugated Mouse Anti His |
| antibody | Secondary Antibody |

TABLE 15
The 8D2H2L15 and 8D2H2L17 antibodies compete
with B7-1 for binding to CTLA4 in ELISA

| | Antigen coating: B7/1-hFc at 0.5 μg/ml | | | | | | | | | | | |
|---|---|---|---|---|---|---|---|---|---|---|---|---|
| Dilution of | 8D2 H2L2 | | | | | | | | | | 8D2 H2L2 | |
| Antibody | (20150327) | | 8D2 H2L15 | | 8D2 H2L17 | | 10D1 | | 11.2.1 | | (20140422) | |
| 5 μg/ml | 0.09 | 0.10 | 0.07 | 0.07 | 0.06 | 0.07 | 0.08 | 0.11 | 0.06 | 0.06 | 0.12 | 0.14 |
| 1:3 | 0.13 | 0.14 | 0.07 | 0.07 | 0.06 | 0.07 | 0.33 | 0.24 | 0.09 | 0.08 | 0.26 | 0.24 |
| 1:9 | 0.29 | 0.26 | 0.07 | 0.09 | 0.08 | 0.08 | 0.71 | 0.78 | 0.33 | 0.30 | 0.45 | 0.49 |
| 1:27 | 0.66 | 0.58 | 0.70 | 1.03 | 0.89 | 0.93 | 1.11 | 1.17 | 1.14 | 1.19 | 1.06 | 1.10 |
| 1:81 | 0.69 | 0.62 | 0.68 | 1.18 | 0.97 | 0.79 | 1.16 | 1.35 | 1.17 | 1.20 | 1.09 | 1.09 |
| 1:243 | 0.66 | 0.64 | 0.75 | 1.13 | 1.05 | 0.99 | 1.27 | 1.48 | 1.30 | 1.31 | 1.19 | 0.99 |
| 1:729 | 0.69 | 0.64 | 0.74 | 1.07 | 1.25 | 1.35 | 1.33 | 1.56 | 1.32 | 1.31 | 1.16 | 1.12 |
| 0 | 0.59 | 0.66 | 0.53 | 1.09 | 1.18 | 1.18 | 1.33 | 1.29 | 1.28 | 1.30 | 1.11 | 1.04 |
| Ligand | CTLA4-mFc at 0.3 μg/ml | | | | | | | | | | | |
| Secondary | HRP Conjugated Mouse Anti His Secondary Antibody | | | | | | | | | | | |
| antibody | | | | | | | | | | | | |

TABLE 16
The 8D2H2L15 and 8D2H2L17 antibodies compete
with B7-2 for binding to CTLA4 in ELISA

| | CTLA4-mFc at 2 μg/ml | | | | | | | | | | | |
|---|---|---|---|---|---|---|---|---|---|---|---|---|
| Dilution of | 8D2H2L2 | | | | | | | | | | 8D2H2L2 | |
| Antibody | 20140422 | | 8D2H2L15 | | 8D2H2L17 | | 10D1 | | 11.2.1 | | 20150327 | |
| 1 μg/ml | 0.05 | 0.05 | 0.05 | 0.05 | 0.05 | 0.05 | 0.05 | 0.05 | 0.04 | 0.04 | 0.05 | 0.05 |
| 1:3 | 0.05 | 0.05 | 0.05 | 0.05 | 0.05 | 0.05 | 0.07 | 0.07 | 0.05 | 0.05 | 0.47 | 0.37 |
| 1:9 | 0.15 | 0.16 | 0.17 | 0.19 | 0.06 | 0.12 | 0.44 | 0.35 | 0.17 | 0.16 | 0.65 | 0.58 |
| 1:27 | 0.55 | 0.59 | 0.42 | 0.48 | 0.50 | 0.57 | 0.73 | 0.70 | 0.57 | 0.57 | 0.79 | 0.70 |
| 1:81 | 0.76 | 0.84 | 0.75 | 0.75 | 0.77 | 0.81 | 0.85 | 0.86 | 0.84 | 0.76 | 0.86 | 0.77 |
| 1:243 | 0.84 | 0.79 | 0.83 | 0.84 | 0.82 | 0.87 | 0.86 | 0.89 | 0.84 | 0.85 | 0.83 | 0.84 |
| 1:729 | 0.77 | 0.76 | 0.94 | 1.00 | 0.97 | 0.98 | 0.99 | 0.91 | 0.87 | 0.85 | 0.82 | 0.80 |
| 0 | 0.77 | 0.78 | 0.92 | 0.97 | 0.81 | 0.82 | 0.76 | 0.96 | 0.91 | 0.80 | 0.80 | 0.76 |
| Ligand | B7/2-His, 0.5 μg/ml | | | | | | | | | | | |
| Secondary | HRP Conjugated Mouse Anti His Secondary Antibody (1:4000) | | | | | | | | | | | |
| antibody | | | | | | | | | | | | |

TABLE 17
The binding efficiency, EC₅₀, of 8D2, 8D2(Re) and the 8D2 humanized
antibodies 8D2H1L1, 8D2H2L2, 8D2H3L3, 8D2H2L15, and 8D2H2L17
to the CTLA4 antigen in competition with B7 obtained by curve
simulation in the analysis by competitive ELISA

| | Antibody EC₅₀ | | 10D1 EC₅₀ | | 11.2.1 EC₅₀ | |
|---|---|---|---|---|---|---|
| | (nM) | | (nM) | | (nM) | |
| | B7-1 | B7-2 | B7-1 | B7-2 | B7-1 | B7-2 |
| 8D2 | 0.44 | 0.208 | — | 0.464 | — | 0.15 |
| 8D2(Re) | 0.514 | 0.153 | — | 0.464 | — | 0.15 |
| 8D2H1L1 | 2.478 | 0.178 | 1.91 | 0.464 | 1.691 | 0.15 |
| 8D2H2L2 | 5.932 | 1.643 | 5.15 | 2.056 | 1.073 | 0.172 |
| 8D2H2L2 | 2.973 | 0.368 | — | — | — | — |
| 8D2H2L2 | 3.118 | 0.301 | — | — | — | — |
| 8D2H3L3 | 2.144 | 0.167 | 5.15 | 2.056 | 1.073 | 0.172 |
| 8D2H2L15 | 1.973 | 0.227 | 4.586 | 0.629 | 2.606 | 0.349 |
| 8D2H2L17 | 1.787 | 0.296 | 4.586 | 0.629 | 2.606 | 0.349 |

Note:
8D2H2L2 was tested in triplicate.

The above results demonstrated that the antibodies 8D2, 8D2 (Re) and the 8D2 humanized antibodies 8D2H1L1, 8D2H2L2, 8D2H3L3, 8D2H2L15, and 8D2H2L17 all can compete with B7 for binding to the CTLA4 antigen. Particularly, 8D2, 8D2(Re), 8D2H1L1, and 8D2H2L2 are stronger than 10D1 in competing with B7-2 for binding to CTLA4, while 8D2H2L17 is stronger than the antibodies 10D1 and 11.2.1 in competing with both of B7-1 and B7-2 for binding to CTLA4.

### Example 9. Analysis of the Biological Activities of the Monoclonal Antibody 8D2 and the Humanized Antibodies 8D2H1L1, 8D2H2L2, 8D2H3L3, 8D2H2L15, and 8D2H2L17 in Cells

To detect the effect of the monoclonal antibody 8D2 and the humanized antibodies 8D2H1L1, 8D2H2L2, 8D2H3L3, 8D2H2L15, and 8D2H2L17, and the control antibodies 10D1 and 11.2.1 on the IL-2 expression of peripheral blood mononuclear cells (PBMC's), peripheral blood from healthy donors was collected into collection tubes containing heparin sodium. PBMC's were obtained as a cell suspension after dilution in PBS and centrifugation on separation medium (at 2550 rpm for 20 min). The cell suspension was added with SEB (1 μg/mL)/PHA (30 μl/ml) and placed in an incubator at 37° C. with saturated humidity containing 5% CO₂ for further culture. Raji lymphocytes and the antibody were added. After co-incubation for 48 hours, PBMC's were washed with PBS twice, and were add to 96 well plates at 10,000 cell/well. Then, the corresponding concentration gradient of the antibodies was added. After inoculation for 20 minutes, Raji cells treated with MMC for 1 hour were added at 10,000 cells/well for co-culture of 72 hours. After co-culture for 72 hours, the cell culture was collected for supernatant and the IL-2 expression profile in the supernatant of the cell co-culture was detected using an ELISA kit following the instructions provided with the kit (Dakewe Co., DKW12-1020-096).

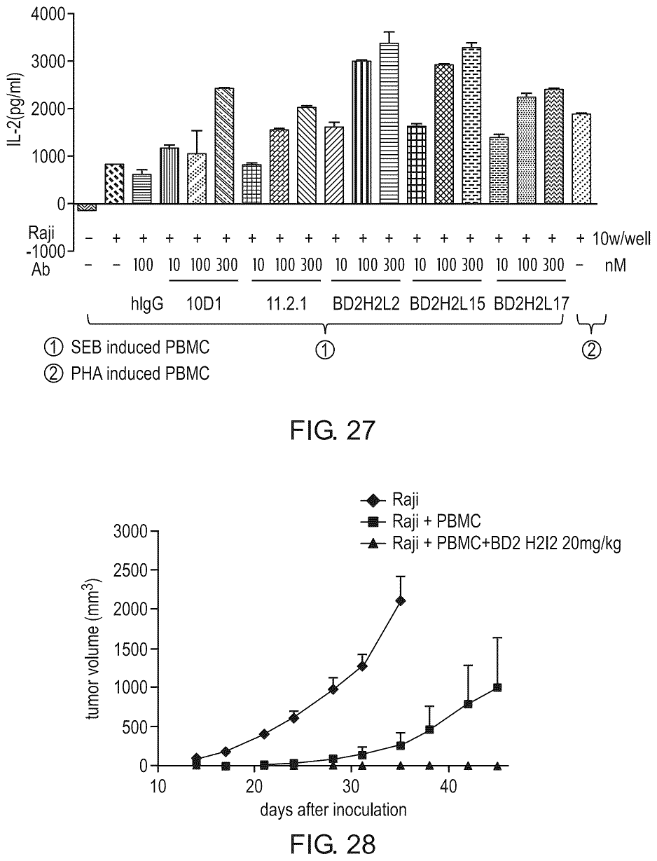

After statistical analysis, the results of the experiments are showed in FIGS. 26 and 27. As compared with the T cell group and the Raji cell group, for the monoclonal antibody 8D2, all its humanized antibodies 8D2H1L1, 8D2H2L2, 8D2H3L3, 8D2H2L15, and 8D2H2L17 can effectively block binding of CTLA4 to B7 and improve the IL-2 expression in T lymphocytes (FIGS. 26 and 27). Particularly, the inventor surprisingly discovered that 8D2H2L2, 8D2H2L15 and 8D2H2L17 are significantly superior than the control antibodies 10D1 and 11.2.1. At the concentration of 10 nM, they achieved an IL-2 level comparable or even better than that achieved by 10D1 or 11.2.1 at the concentration of 100 nM. Thus, the antibodies of the present invention can increase the level of IL-2 at a lower concentration, e.g., about 10 nM.

### Example 10. The In Vivo Anti-Tumor Activity of the Monoclonal Antibody 8D2H2L2

The in vivo anti-tumor activity of 8D2H2L2 was evaluated using the hu-SCID-raji animal model. Human peripheral blood mononuclear cells (PBMC's) were isolated using the Ficoll reagent, and activated using SEB at 1 μg/ml for 3 days. Then, 1.25×10⁶ activated PBMC's were mixed with 5×10⁶ raji Burkitt lymphoma cells and 8D2H2L2 (20 mg/kg), and were injected subcutaneously on the back of SCID-beige mice. At the same time, an isotype control group was set up, 5 animals per group. Subsequently, a dose of 20 mg/kg was administered by intravenous injection once a week for three consecutive weeks. The tumor volume was measured twice a week until the end of the experiments or when the tumor volume reached 1000 mm³.

As shown in FIG. 28, 8D2H2L2 could notably inhibit tumor growth in the hu-SCID-raji model. This result indicated that this antibody can be used clinically to treat lymphoma.

While the specific embodiments of the invention have been described in detail, those skilled in the art, in light of the teaching disclosed in the specification, will understand that various changes and modifications can be made to the details, all of which fall into the protection scope of the present invention. The full scope of the invention is set forth in the appended claims and any equivalents thereof.

1. A monoclonal antibody or antigen binding fragment thereof, wherein the monoclonal antibody or antigen binding fragment thereof binds to human CTLA4 and comprises a heavy chain variable region,
wherein the heavy chain variable region comprises:
a. an HCDR1 comprising the amino acid sequence of SEQ ID NO: 27,
b. an HCDR2 comprising the amino acid sequence of SEQ ID NO: 28, and
c. an HCDR3 comprising the amino acid sequence of SEQ ID NO: 29.

2. The monoclonal antibody or antigen binding fragment thereof according to claim 1, wherein
the amino acid sequence of the heavy chain variable region (VH) of the monoclonal antibody is selected from the group consisting of: SEQ ID NO: 6, SEQ ID NO: 10, SEQ ID NO: 14, SEQ ID NO: 18, and any of SEQ ID NO: 6, SEQ ID: 10, and SEQ ID NO: 14, wherein the methionine at position 18 in SEQ ID NO: 6, SEQ ID NO: 10, and SEQ ID NO: 14 is substituted with an amino acid selected from the group consisting of: leucine, valine, isoleucine, and alanine.

3. The monoclonal antibody or antigen binding fragment thereof according to claim 2, wherein the monoclonal antibody comprises:
(1) the VH as set forth in SEQ ID NO: 6 and the VL as set forth in SEQ ID NO: 8;
(2) the VH as set forth in SEQ ID NO: 10 and the VL as set forth in SEQ ID NO: 12;
(3) the VH as set forth in SEQ ID NO: 14 and the VL as set forth in SEQ ID NO: 16;
(4) the VH as set forth in SEQ ID NO: 18 and the VL as set forth in SEQ ID NO: 20;
(5) the VH as set forth in SEQ ID NO: 14 and the VL as set forth in SEQ ID NO: 22; or
(6) the VH as set forth in SEQ ID NO: 14 and the VL as set forth in SEQ ID NO: 24;
(7) the VH as set forth in SEQ ID NO: 6, wherein the methionine at amino acid position 18 is substituted with leucine, valine, isoleucine, or alanine, and the VL as set forth in SEQ ID NO: 8;
(8) the VH as set forth in SEQ ID NO: 10, wherein the methionine at amino acid position 18 is substituted with leucine, valine, isoleucine, or alanine, and the VL as set forth in SEQ ID NO: 12;
(9) the VH as set forth in SEQ ID NO: 14, wherein the methionine at amino acid position 18 is substituted with leucine, valine, isoleucine, or alanine, and the VL as set forth in SEQ ID NO: 16;
(10) the VH as set forth in SEQ ID NO: 14, wherein the methionine at amino acid position 18 is substituted with leucine, valine, isoleucine, or alanine, and the VL as set forth in SEQ ID NO: 22; and
(11) the VH as set forth in SEQ ID NO: 14, wherein the methionine at amino acid position 18 is substituted with leucine, valine, isoleucine, or alanine, and the VL as set forth in SEQ ID NO: 24.

4. The monoclonal antibody or antigen binding fragment thereof of claim 3, wherein the methionine at amino acid position 18 of SEQ ID NO: 6, SEQ ID NO: 10, or SEQ ID NO: 14 is substituted with leucine.

5. The monoclonal antibody or antigen binding fragment thereof according to claim 1, wherein the monoclonal antibody or antigen binding fragment thereof is selected from the group consisting of: an Fab, an Fab′, an F(ab′).sub.2, an Fd, an Fv, a dAb, a complementarity determining region fragment, a single chain antibody, a humanized antibody, a chimeric antibody, and a diabody.

6. The monoclonal antibody or antigen binding fragment thereof according to claim 1, wherein the monoclonal antibody or antigen binding fragment thereof binds to the human CTLA4 protein with a K_{D} less than about 10⁻⁵ M.

7. The monoclonal antibody or antigen binding fragment thereof according to claim 1, wherein the monoclonal antibody or antigen binding fragment thereof comprises non-CDR regions and the non-CDR regions are from a human.

8. A conjugate comprising the monoclonal antibody or antigen binding fragment thereof according to claim 1 and a conjugated moiety, wherein the conjugated moiety is a detectable label; specifically, the conjugated moiety is a radioisotope, a fluorescent substance, a luminescent substance, a chromogenic substance, or an enzyme.

9. An in vitro method of relieving CTLA4 immunosuppression comprising contacting cells with an effective amount of the monoclonal antibody or antigen binding fragment thereof according to claim 1, where immunosuppression is measured by blocking binding of human CTLA4 to human B7 or by measuring an increase in IL-2 expression in T lymphocytes.

10. A method of diagnosing a tumor, comprising the step of administrating an effective amount of a monoclonal antibody or antigen binding fragment thereof to a sample of the tumor obtained from a patient and assessing whether the sample of the tumor binds to the monoclonal antibody or antigen binding fragment thereof, wherein the sample of the tumor is a biopsy of the patient, wherein the tumor is selected from the group consisting of: a melanoma, a kidney tumor, a prostate cancer, a bladder cancer, a colorectal cancer, a cancer of gastrointestinal tract, and a liver cancer, and wherein the monoclonal antibody or antigen binding fragment thereof comprises the complementary determining regions (CDR's) selected from the following:
a. an HCDR1 comprising the amino acid sequence of SEQ ID NO: 27,
b. an HCDR2 comprising the amino acid sequence of SEQ ID NO: 28, and
c. an HCDR3 comprising the amino acid sequence of SEQ ID NO: 29, and wherein the monoclonal antibody or antigen binding fragment thereof is linked to a detectable compound.

11. An isolated nucleic acid molecule comprising a nucleic acid sequence that encodes a heavy chain variable region of an antibody, wherein the heavy chain variable region of the antibody comprises: CDRs with the amino acid sequences of SEQ ID NOs: 27-29.

12. (canceled)

13. A vector comprising the isolated nucleic acid molecule of claim 11.

14. A host cell comprising the vector of claim 13.

15. A host cell comprising the isolated nucleic acid molecule of claim 11.

16. The isolated nucleic acid molecule of claim 11, wherein the nucleic acid sequence encodes the heavy chain variable region of an antibody, and the heavy chain variable region of the antibody has the amino acid sequence as set forth in SEQ ID NO: 6, SEQ ID NO: 10, SEQ ID NO: 14, or SEQ ID NO: 18, wherein the methionine at position 18 in SEQ ID NO: 6, SEQ ID NO: 10, and SEQ ID NO: 14 is independently substituted with an amino acid selected from the group consisting of: leucine, valine, isoleucine, and alanine.

17. A host cell comprising the isolated nucleic acid molecule of claim 16.

18. A vector comprising the isolated nucleic acid molecule of claim 16.

19. A host cell comprising the vector of claim 18.

20-34. (canceled)

35. A method of preparing the monoclonal antibody or antigen binding fragment thereof comprising culturing the host cell of claim 17 under suitable conditions, collecting a supernatant from the cell culture, and purifying the supernatant to recover the monoclonal antibody or antigen binding fragment thereof.


SEQUENCE LISTING (34 sequences)

SEQ ID NO: 1 - DNA, 375 bp, synthetic construct
  misc_feature 1..375: note = the nucleic acid sequence encoding the CTLA4ECD protein
  source 1..375: mol_type = other DNA
gcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgac

SEQ ID NO: 2 - protein, 125 aa, synthetic construct
  REGION 1..125: note = the amino acid sequence of the CTLA4ECD protein
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 3 - protein, 364 aa, synthetic construct
  REGION 1..364: note = the amino acid sequence of the CTLA4ECD-mFc chimeric protein
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDENLYFQGPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - DNA, 1092 bp, synthetic construct
  misc_feature 1..1092: note = the nucleic acid sequence encoding the CTLA4ECD-mFc chimeric protein
  source 1..1092: mol_type = other DNA
gcaatgcatgtcgcacagcctgcagtggtcctggcaagctccaggggaatcgctagcttcgtgtgcgaatacgcttccccaggcaaggcaaccgaggtccgggtgacagtcctgagacaggccgacagccaggtgacagaagtctgcgccgctacttatatgatgggcaacgagctgacctttctggacgatagcatttgtaccgggacatctagtggaaaccaagtgaatctgaccatccagggcctgcgcgctatggacacagggctgtacatttgtaaagtggagctgatgtatccccctccatactatctgggaatcggcaacgggacccagatctacgtgattgatcctgaaccatgccccgactccgatgagaatctgtatttccagggaccacgaggccccacaattaagccatgtcccccttgcaaatgtcctgcaccaaacctgctgggaggaccaagcgtgttcatctttccacccaagatcaaggacgtgctgatgatctcactgagccccattgtgacctgcgtggtcgtggacgtgagcgaggacgatcctgatgtgcagatcagttggttcgtcaacaatgtggaagtccacacagctcagactcagacccatagggaggattacaatagtactctgcgcgtcgtgtcagcactgcccattcagcaccaggactggatgagcggcaaggagttcaagtgcaaagtgaacaacaaggatctgcccgcacctatcgagagaactatttccaagcctaaagggtctgtgagggccccacaggtgtatgtcctgcctccacccgaggaagagatgactaagaaacaggtgacactgacttgtatggtcaccgacttcatgcccgaagatatctacgtggagtggactaacaatgggaagaccgaactgaactataaaaatacagagcctgtgctggactcagatggaagctactttatgtatagcaagctgcgagtggaaaagaaaaactgggtcgagcggaacagctactcttgtagtgtggtccacgaagggctgcataatcaccacaccactaaatcattctcccgaactccaggcaaa

SEQ ID NO: 5 - DNA, 345 bp, synthetic construct
  misc_feature 1..345: note = the nucleic acid sequence encoding the heavy chain variable domain of the monoclonal antibody 8D2
  source 1..345: mol_type = other DNA
gaggtgaaactggacgaaactggcggggggctggtgcagcccggacgacctatgaagctgtcatgcgtcgccagcggcttcacctttagcgacaactggatgaattgggtgaggcagagcccagagaaggggctggaatggctggctcagatccgcaacaaaccctacaattatgagacctactattctgacagtgtgaagggccggttcacaatttccagagacgattctaaaagctccgtctacctgcagatgaacaatctgagaggcgaagatatggggatctactattgcacagcacagttcgcttattggggacagggcactctggtcacagtctccgcc

SEQ ID NO: 6 - protein, 115 aa, synthetic construct
  REGION 1..115: note = the amino acid sequence of the heavy chain variable domain of the monoclonal antibody 8D2
EVKLDETGGGLVQPGRPMKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 7 - DNA, 318 bp, synthetic construct
  misc_feature 1..318: note = the nucleic acid sequence encoding the light chain variable domain of the monoclonal antibody 8D2
  source 1..318: mol_type = other DNA
gacattcagatgacacagagtcctgcttccctgagtgcctcagtgggggagaccgtcacaatcacttgcggcacctctgaaaacatctacggcgggctgaattggtatcagcggaagcagggcaaaagtccccagctgctgatcttcggagcaacaaacctggccgacggcatgagctcccggtttagcgggtccggatctggcagacagtacagcctgaagatttctagtctgcacccagacgatgtggctacttactattgccagaatgtcctgaggagtcccttcacctttgggtcaggaacaaagctggagatc

SEQ ID NO: 8 - protein, 106 aa, synthetic construct
  REGION 1..106: note = the amino acid sequence of the light chain variable domain of the monoclonal antibody 8D2
DIQMTQSPASLSASVGETVTITCGTSENIYGGLNWYQRKQGKSPQLLIFGATNLADGMSSRFSGSGSGRQYSLKISSLHPDDVATYYCQNVLRSPFTFGSGTKLEI

SEQ ID NO: 9 - DNA, 345 bp, synthetic construct
  misc_feature 1..345: note = the nucleic acid sequence encoding the heavy chain variable domain of the monoclonal antibody 8D2H1L1
  source 1..345: mol_type = other DNA
gaagtgcagctggtcgagtccggggggggcctggtgcagccaggaggatcaatgcgactgagctgcgccgcttccggcttcaccttcagcgacaactggatgaattgggtcaggcaggcaccaggaaagggactggagtggctggcacagatccgcaacaaaccttacaactacgaaacttactacagcgactccgtgaaggggcggttcaccatttctagagacgattctaaaaacagtgtgtacctgcagatgaatagcctgaagaccgaggatacaggagtctactattgtaccgcacagtttgcttattgggggcagggcactctggtgacagtctcttca

SEQ ID NO: 10 - protein, 115 aa, synthetic construct
  REGION 1..115: note = the amino acid sequence of the light chain variable domain of the monoclonal antibody 8D2H1L1
EVQLVESGGGLVQPGGSMRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 11 - DNA, 321 bp, synthetic construct
  misc_feature 1..321: note = the nucleic acid sequence encoding the light chain variable domain of the monoclonal antibody 8D2H1L1
  source 1..321: mol_type = other DNA
gacattcagatgactcagagcccttcaagcctgtccgcatctgtgggcgaccgagtcaccatcacatgcagaacctccgagaacatctacggcgggctgaattggtatcagcgaaagcaggggaaaagtcccaagctgctgatctacggggcaacaaacctggccagcggaatgagctccagattcagtggatcaggcagcgggacagattatactctgaaaatttctagtctgcacccagacgatgtggcaacctactattgccagaatgtcctgaggtcacccttcacctttggaagcggcacaaaactggagatcaag

SEQ ID NO: 12 - protein, 107 aa, synthetic construct
  REGION 1..107: note = the amino acid sequence of the light chain variable domain of the monoclonal antibody 8D2H1L1
DIQMTQSPSSLSASVGDRVTITCRTSENIYGGLNWYQRKQGKSPKLLIYGATNLASGMSSRFSGSGSGTDYTLKISSLHPDDVATYYCQNVLRSPFTFGSGTKLEIK

SEQ ID NO: 13 - DNA, 345 bp, synthetic construct
  misc_feature 1..345: note = the nucleic acid sequence encoding the heavy chain variable domain of the monoclonal antibody 8D2H2L2, 8D2H2L15 or 8D2H2L17
  source 1..345: mol_type = other DNA
gaagtgcagctggtcgagtccggggggggcctggtgcagccaggaggatcaatgcgactgagctgcgccgcttccggcttcaccttcagcgacaactggatgaattgggtcaggcaggcaccaggaaagggactggagtggctggcacagatccgcaacaaaccttacaactacgaaacttactacagcgcctccgtgaaggggcggttcaccatttctagagacgattctaaaaacagtgtgtacctgcagatgaatagcctgaagaccgaggatacaggagtctactattgtaccgcacagtttgcttattgggggcagggcactctggtgacagtctcttca

SEQ ID NO: 14 - protein, 115 aa, synthetic construct
  REGION 1..115: note = the amino acid sequence of the heavy chain variable domain of the monoclonal antibody 8D2H2L2, 8D2H2L15 or 8D2H2L17
EVQLVESGGGLVQPGGSMRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 15 - DNA, 321 bp, synthetic construct
  misc_feature 1..321: note = the nucleic acid sequence encoding the light chain variable domain of the monoclonal antibody 8D2H2L2
  source 1..321: mol_type = other DNA
gacattcagatgactcagagcccttcaagcctgagtgcctcagtgggagaccgggtcaccatcacatgcagaaccagcgagaacatctacggcggcctgaactggtatcagcgaaagccaggcaagagccccaagctgctgatctacggggcaaccaacctggcctctggagtgagctccagattcagcggcagcggctctgggaccgactatactctgaccatttctagtctgcagcctgaagatgtggcaacatactattgccagaatgtcctgaggtccccattcacctttggatctggcaccaagctggagatcaag

SEQ ID NO: 16 - protein, 107 aa, synthetic construct
  REGION 1..107: note = the amino acid sequence of the light chain variable domain of themonoclonal antibody 8D2H2L2
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIK

SEQ ID NO: 17 - DNA, 345 bp, synthetic construct
  misc_feature 1..345: note = the nucleic acid sequence encoding the heavy chain variable domain of the monoclonal antibody 8D2H3L3
  source 1..345: mol_type = other DNA
gaggtgcagctggtcgagtctggaggcggcctggtgcagcccggcgggtcactgcgactgagctgcgccgcttccggcttcaccttcagcgacaactggatgaattgggtgaggcaggcacccgggaaggggctggagtgggtcgctcagatccgcaacaaaccttacaattatgagacagaatacgcagcctctgtgaaggggcggttcactattagtagagacgatagcaagaacagcgcctatctgcagatgaatagcctgaagaccgaagatacagccgtctactattgtacagctcagtttgcatactggggccagggaactctggtgaccgtcagctcc

SEQ ID NO: 18 - protein, 115 aa, synthetic construct
  REGION 1..115: note = the amino acid sequence of the heavy chain variable domain of the monoclonal antibody 8D2H3L3
EVQLVESGGGLVQPGGSLRLSCAASGFTFSDNWMNWVRQAPGKGLEWVAQIRNKPYNYETEYAASVKGRFTISRDDSKNSAYLQMNSLKTEDTAVYYCTAQFAYWGQGTLVTVSS

SEQ ID NO: 19 - DNA, 321 bp, synthetic construct
  misc_feature 1..321: note = the nucleic acid sequence encoding the light chain variable domain of the monoclonal antibody 8D2H3L3
  source 1..321: mol_type = other DNA
gacattcagatgactcagagcccttcttctctgtccgcatctgtgggagaccgggtcaccatcacatgcagagccagcgagaacatctacggcggcctgaactggtatcagcagaagccaggcaaagctcccaagctgctgatctacggagcaacctccctggcatctggagtgccatcccggttcagtggatcaggcagcgggaccgactatactctgaccattagctccctgcagcctgaagacttcgccacatactattgccagaacgtgctgaggtccccattcacctttggatctggcaccaagctggagatcaag

SEQ ID NO: 20 - protein, 107 aa, synthetic construct
  REGION 1..107: note = the amino acid sequence of the light chain variable domain of the monoclonal antibody 8D2H3L3
DIQMTQSPSSLSASVGDRVTITCRASENIYGGLNWYQQKPGKAPKLLIYGATSLASGVPSRFSGSGSGTDYTLTISSLQPEDFATYYCQNVLRSPFTFGSGTKLEIK

SEQ ID NO: 21 - DNA, 321 bp, synthetic construct
  misc_feature 1..321: note = the nucleic acid sequence encoding the light chain variable domain of the monoclonal antibody 8D2H2L15
  source 1..321: mol_type = other DNA
gacatccagatgactcagtctcccagctccctgtccgcttctgtgggcgatcgggtcactatcacctgtagaaccagcgagaacatttacggcggactgaattggtatcagaggaagcccgggaaaagtcctaagctgctgatctacggagcaacaaacctggcctccggcgtgtctagtcgcttcagtggatcaggcagcgggaccgactatacactgactatttcaagcctgcagccagaggatgtggccacatactattgccagaatgtcctgagccggcaccccggatttggctcagggaccaaactggaaattaag

SEQ ID NO: 22 - protein, 107 aa, synthetic construct
  REGION 1..107: note = the amino acid sequence of the light chain variable domain of the monoclonal antibody 8D2H2L15
DIQMTQSPSSLSASVGDRVTITCRTSENIYGGLNWYQRKPGKSPKLLIYGATNLASGVSSRFSGSGSGTDYTLTISSLQPEDVATYYCQNVLSRHPGFGSGTKLEIK

SEQ ID NO: 23 - DNA, 321 bp, synthetic construct
  misc_feature 1..321: note = the nucleic acid sequence encoding the light chain variable domain of the monoclonal antibody 8D2H2L17
  source 1..321: mol_type = other DNA
gacatccagatgactcagtcacccagctccctgagtgcttcagtgggcgatcgggtcactatcacctgtagaaccagcgagaacatttacggcggactgaattggtatcagaggaagcccgggaaaagccctaagctgctgatctacggagcaacaaacctggcctccggcgtgtctagtcgcttcagcggcagcggctctggaaccgactatacactgactatttcaagcctgcagccagaggatgtggccacatactattgccagaatgtcctgtcctctcgacccggatttggcagtgggaccaaactggaaattaag

SEQ ID NO: 24 - protein, 107 aa, synthetic construct
  REGION 1..107: note = the amino acid sequence of the light chain variable domain of the monoclonal antibody 8D2H2L17
DIQMTQSPSSLSASVGDRVTITCRTSENIYGGLNWYQRKPGKSPKLLIYGATNLASGVSSRFSGSGSGTDYTLTISSLQPEDVATYYCQNVLSSRPGFGSGTKLEIK

SEQ ID NO: 25 - DNA, 636 bp, synthetic construct
  misc_feature 1..636: note = the nucleic acid sequence encoding the CTLA4 protein
  source 1..636: mol_type = other DNA
atgggcgtcctgctgactcagagaaccctgctgtccctggtgctggcactgctgtttccttcaatggcttcaatggctatgcatgtggctcagccagcagtggtcctggcaagctccagggggatcgccagtttcgtgtgcgagtacgcctcacctggaaaggctacagaagtccgggtgactgtcctgagacaggctgactctcaggtgaccgaggtctgcgccgctacatatatgatgggcaacgaactgacctttctggacgattccatttgtactggcacctctagtgggaaccaagtgaatctgactatccagggactgcgagcaatggacaccggactgtacatttgcaaagtggagctgatgtatccccctccatactatctgggcatcgggaatggaacacagatctacgtgattgatcccgaaccttgtccagacagcgatttcctgctgtggattctggcagccgtgtcaagcggcctgttcttttatagctttctgctgactgccgtctccctgtctaagatgctgaagaaacgatcccccctgaccacaggggtggtcgtgaaaatgccacctaccgagcccgagtgcgaaaaacagttccagccatactttatccctatcaat

SEQ ID NO: 26 - protein, 212 aa, synthetic construct
  REGION 1..212: note = the amino acid sequence of the CTLA4 protein
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVVVKMPPTEPECEKQFQPYFIPIN

SEQ ID NO: 27 - protein, 8 aa, synthetic construct
  REGION 1..8: note = the amino acid sequence of CDR
GFTFSDNW

SEQ ID NO: 28 - protein, 10 aa, synthetic construct
  REGION 1..10: note = the amino acid sequence of CDR
IRNKPYNYET

SEQ ID NO: 29 - protein, 6 aa, synthetic construct
  REGION 1..6: note = the amino acid sequence of CDR
TAQFAY

SEQ ID NO: 30 - protein, 6 aa, synthetic construct
  REGION 1..6: note = the amino acid sequence of CDR
ENIYGG

SEQ ID NO: 31 - skipped

SEQ ID NO: 32 - protein, 9 aa, synthetic construct
  REGION 1..9: note = the amino acid sequence of CDR
QNVLRSPFT

SEQ ID NO: 33 - protein, 9 aa, synthetic construct
  REGION 1..9: note = the amino acid sequence of CDR
QNVLSRHPG

SEQ ID NO: 34 - protein, 9 aa, synthetic construct
  REGION 1..9: note = the amino acid sequence of CDR
QNVLSSRPG